SEQUENCE LISTING (45 sequences)

SEQ ID NO: 1 - protein, 20 aa, Human papillomavirus type 16
QLYKTCKQAGTCPPDIIPKV

SEQ ID NO: 2 - protein, 20 aa, Human papillomavirus type 52
QLYQTCKASGTCPPDVIPKV

SEQ ID NO: 3 - protein, 20 aa, Human papillomavirus type 51
QLYSTCKAAGTCPPDVVNKV

SEQ ID NO: 4 - protein, 20 aa, Human papillomavirus type 6
QLYQTCKLTGTCPPDVIPKV

SEQ ID NO: 5 - protein, 20 aa, Human papillomavirus type 11
QLYQTCKATGTCPPDVIPKV

SEQ ID NO: 6 - protein, 20 aa, Human papillomavirus type 31
QLYQTCKAAGTCPSDVIPKI

SEQ ID NO: 7 - protein, 20 aa, Human papillomavirus type 45
DLYRTCKQSGTCPPDVINKV

SEQ ID NO: 8 - protein, 20 aa, Human papillomavirus type 16
GGLGIGTGSGTGGRTGYIPL

SEQ ID NO: 9 - protein, 20 aa, Human papillomavirus type 6
GGLGIGTGSGTGGRTGYVPL

SEQ ID NO: 10 - protein, 20 aa, Human papillomavirus type 31
GGLGIGSGSGTGGRTGYVPL

SEQ ID NO: 11 - protein, 20 aa, Human papillomavirus type 33
GGLGIGTGSGSGGRTGYVPI

SEQ ID NO: 12 - protein, 20 aa, Human papillomavirus type 45
GGLGIGTGSGSGGRTGYVPL

SEQ ID NO: 13 - protein, 20 aa, Human papillomavirus type 11
GGLGIGTGAGSGGRAGYIPL

SEQ ID NO: 14 - protein, 20 aa, Human papillomavirus type 35
GGLGIGSGSGTGGRSGYVPL

SEQ ID NO: 15 - protein, 20 aa, Human papillomavirus type 52
GGLGIGTGAGSGGRAGYVPL

SEQ ID NO: 16 - protein, 20 aa, Human papillomavirus type 6
EPVAPSDPSIVSLIEESAII

SEQ ID NO: 17 - protein, 20 aa, Human papillomavirus type 52
EPIGPLEPSIVSMIEETTFI

SEQ ID NO: 18 - protein, 20 aa, Human papillomavirus type 31
DPVGPLDPSIVSLVEESGIV

SEQ ID NO: 19 - protein, 20 aa, Human papillomavirus type 16
DPVGPSDPSIVSLVEETSFI

SEQ ID NO: 20 - protein, 20 aa, Human papillomavirus type 58
DTVGPLDSSIVSLIEESSFI

SEQ ID NO: 21 - protein, 20 aa, Human papillomavirus type 45
EPVGPTDPSIVTLVEDSSVV

SEQ ID NO: 22 - protein, 13 aa, Human papillomavirus type 16
LVEETSFIDAGAP

SEQ ID NO: 23 - protein, 13 aa, Human papillomavirus type 18
LIEDSSVVTSGAP

SEQ ID NO: 24 - protein, 14 aa, Artificial Sequence
  Synthetic L2 peptide
QLYKTCPPDIIPKV

SEQ ID NO: 25 - protein, 14 aa, Artificial Sequence
  Synthetic L2 peptide
QLYKTCPPDVIPKV

SEQ ID NO: 26 - protein, 20 aa, Artificial Sequence
  Synthetic L2 peptide
QLYKTCKQAGTCPPDVIPKV

SEQ ID NO: 27 - protein, 20 aa, Artificial Sequence
  Synthetic L2 peptide
QLYSTCKAAGTCPPDVINKV

SEQ ID NO: 28 - protein, 20 aa, Artificial Sequence
  Synthetic L2 peptide
DLYRTCKQSGTCPPDVIPKV

SEQ ID NO: 29 - protein, 8 aa, Human papillomavirus type 16
GGLGIGTG

SEQ ID NO: 30 - protein, 21 aa, Artificial Sequence
  Synthetic L2 peptide
QLYKTPPDIIPKVGGLGIGTG

SEQ ID NO: 31 - protein, 21 aa, Artificial Sequence
  Synthetic L2 peptide
QLYKTPPDVIPKVGGLGIGTG

SEQ ID NO: 32 - protein, 5 aa, Human papillomavirus type 16
GGLGI

SEQ ID NO: 33 - protein, 20 aa, Human papillomavirus type 5
HIYQTCKQAGTCPPDVINKV

SEQ ID NO: 34 - protein, 20 aa, Human papillomavirus type 56
QLYKTCKLSGTCPEDVVNKI

SEQ ID NO: 35 - protein, 20 aa, Human papillomavirus type 5
GGLGIGTGSGTGGRTGYVPL

SEQ ID NO: 36 - protein, 492 aa, Artificial sequence
  Chimeric L1/L2 peptide
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSGGLGIGTGSGTGGRTGYVPLNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQ

SEQ ID NO: 37 - protein, 492 aa, Artificial sequence
  Chimeric L1/L2 peptide
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDGGLGIGTGSGTGGRTGYVPLAAPAENKDPYDKLKFWNVDLKEKFSLDLDQYPLGRKFLVQ

SEQ ID NO: 38 - protein, 512 aa, Artificial sequence
  Chimeric L1/L2 peptide
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSQLYQTCKATGTCPPDVIPKVNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDGGLGIGTGSGTGGRTGYVPLAAPAENKDPYDKLKFWNVDLKEKFSLDLDQYPLGRKFLVQ

SEQ ID NO: 39 - protein, 491 aa, Artificial sequence
  Chimeric L1/L2 peptide
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHGGLGIGTGSGTGGRTGYVPLTPPAPKEDPLKKYTFWEVNLKEKFSADLDQFPLGRKFLLQ

SEQ ID NO: 40 - protein, 491 aa, Artificial sequence
  Chimeric L1/L2 peptide
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAAQLYQTCKATGTCPPDVIPKVNAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQ

SEQ ID NO: 41 - protein, 491 aa, Artificial sequence
  Chimeric L1/L2 peptide
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPQLYQTCKATGTCPPDVIPKVDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQ

SEQ ID NO: 42 - protein, 511 aa, Artificial sequence
  Chimeric L1/L2 peptide
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAAQLYQTCKATGTCPPDVIPKVNAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHGGLGIGTGSGTGGRTGYVPLTPPAPKEDPLKKYTFWEVNLKEKFSADLDQFPLGRKFLLQ

SEQ ID NO: 43 - protein, 492 aa, Artificial sequence
  Chimeric L1/L2 peptide
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSQLYQTCKATGTCPPDVIPKVNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQ

SEQ ID NO: 44 - protein, 492 aa, Artificial sequence
  Chimeric L1/L2 peptide
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACVGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPPSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDQLYQTCKATGTCPPDVIPKVAAPAENKDPYDKLKFWNVDLKEKFSLDLDQYPLGRKFLVQ

SEQ ID NO: 45 - protein, 491 aa, Artificial sequence
  Chimeric L1/L2 peptide
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAAGGLGIGTGSGTGGRTGYVPLNAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQ

(19) United States
(12) Patent Application Publication
(10) Pub. No.: US 2012/0087937 A1
(43) Pub. Date: Apr. 12, 2012

(54) NOVEL COMPOSITIONS

(76) Inventors: Brigitte Desiree Alberte Colau, Rixensart (BE); Najoua Dendouga, Rixensart (BE); Sandra Giannini, Rixensart (BE); Nicolas Pierri Fernand Lecrenier, Wavre (BE); Guy Jean Marie Fernand Pierre Baudoux, King of Prussia, PA (US)

(21) Appl. No.: 13/378,446

(22) PCT Filed: Jun. 24, 2010
(86) PCT No.: PCT/EP10/59024

§ 371 Date: Dec. 15, 2011

Related U.S. Application Data
(60) Provisional application No. 61/220,358, filed on Jun. 25, 2009.
(60) Provisional application No. 61/239,880, filed on Sep. 4, 2009.
(60) Provisional application No. 61/322,102, filed on Apr. 8, 2010.

(51) Int. Cl. A61K 39/12; A61P 37/04; C12P 21/02; A61P 31/20; C07K 19/00; C12N 15/62

(52) U.S. Cl. 424/186.1; 530/350; 536/23.4; 435/69.3

(57) ABSTRACT

This disclosure provides novel human papillomavirus (HPV) protein constructs and their use in the prevention of HPV disease. The constructs are chimeric proteins comprising L1 proteins with an HPV L2 peptide inserted in to the L1 protein. Such chimeric proteins may be formulated into immunogenic e.g., vaccine compositions, and optionally formulated with HPV L1 VLP based vaccines.

### BACKGROUND

The present disclosure relates to the field of human vaccines. More particularly, the present disclosure relates to pharmaceutical and immunogenic compositions, for the prevention or treatment of human papillomavirus (HPV) infection or disease.

Papillomaviruses are small, highly species specific, DNA tumour viruses. Human papillomaviruses are DNA viruses that infect basal epithelial (skin or mucosal) cells. Over 100 individual human papillomavirus (HPV) genotypes have been described. HPVs are generally specific either for the squamous epithelium of the skin (e.g. HPV-1 and -2) or mucosal surfaces (e.g. HPV-6 and -11) and usually cause benign tumours (warts) that persist for several months or years.

Persistent infection with an oncogenic human papillomavirus (HPV) type is a necessary cause of cervical cancer, the second most common cause of cancer death among women worldwide. There is international consensus that “high-risk” genotypes, including genotypes 16, 18, 31, 33, 35, 39, 45, 51, 52, 56, 58, 59 and 66, can lead to cervical cancer and are associated with other mucosal anogenital and head and neck cancers. Globally, HPV-16 and HPV-18 are the predominant oncogenic types, cumulatively accounting for over 70-80% of all invasive cervical cancer cases.

Infections with other genotypes, termed “low-risk,” can cause benign or low-grade cervical tissue changes and genital warts (condyloma acuminata), which are growths on the cervix, vagina, vulva and anus in women and the penis, scrotum or anus in men. They also cause epithelial growths over the vocal cords of children and adults (juvenile respiratory papillomatosis or recurrent respiratory papillomatosis) that require surgical intervention.

Two prophylactic HPV vaccines have recently been licensed in many countries. Both use virus-like particles (VLPs) comprised of recombinant L1 capsid proteins of individual HPV types to prevent HPV-16 and -18 cervical precancerous lesions and cancers. Cervarix™ (GlaxoSmithKline Biologicals) contains HPV-16 and -18 VLPs produced in Trichoplusia ni cell substrate using a baculovirus expression vector system and formulated with the immunostimulant 3-O-desacyl-4′-monophosphoryl lipid A (MPL) and aluminium hydroxide salt. Gardasil™ (Merck) contains HPV-16 and -18 VLPs produced in the yeast Saccharomyces cerevisiae and formulated with amorphous aluminium hydroxyphosphate sulphate salt. In addition, Gardasil™ contains VLPs from non-oncogenic types HPV-6 and -11, which are implicated in 75-90% of genital warts. For both vaccines, specific protection against infection with oncogenic types HPV-16 and HPV-18 and associated precancerous lesions has been demonstrated in randomised clinical trials.

The list of oncogenic HPV types which are responsible for causing cervical cancer includes at least HPV types 16, 18, 31, 33, 35, 39, 45, 51, 52, 56, 58, 59, 66, 68 and 73 found in cervical cancer (Mandavi et al, 2005; Quint et al., 2006).

The existing vaccines are able to provide specific protection against infection and/or disease by some of these HPV types and to varying degrees. However it would be potentially beneficial to have a vaccine which either contains antigens from other HPV types in order to further improve the coverage against all of the cervical cancer causing HPV types or would elicit a broad cross protection against related and non-related HPV types. It would be potentially beneficial to have a vaccine which is further effective against skin cancer causing HPV types such as HPV 5 or HPV 8 or HPV 38 or any combination of two or more of these.

In addition to the currently approved L1 VLP vaccines, peptides of L2 have been proposed for use in an HPV vaccine for example in WO 2003/097673, WO 2004/052395, WO 2006/083984, WO 2009/001867, Kondo et al 2008 J Med Virol 80, 841-846, Kondo et al 2006 Virology 358, 266-272, Schellenbacher et al 2009 25^{th} International Papillomavirus Conference 8-14^{th} May, Malmo, Sweden, Coursaget et al, 25^{th} International Papillomavirus Conference 8-14^{th} May, Malmo, Sweden, Slupetzky et al 2007 Vaccine 25, 2001-2010, Xu et al 2006 Arch Virol 151, 2133-2148, Gambhira et al 2007 J Virol 81, 13927-13931, Alphs et al 2008 PNAS 105, 5850-5, Kawana et al 2003 Vaccine 21, 4256-60, Kawana et al 2001 Vaccine 19, 1496-1502.

### BRIEF SUMMARY

The present disclosure relates to an improved vaccine against human papilloma virus which contains antigens which provides protection against additional cancer causing HPV types and/or low risk HPV types associated with genital warts. The improved vaccines contain chimeric L1 polypeptides into which at least one peptide comprising an epitope of an L2 polypeptide is inserted.

In an embodiment of the invention, there is provided a human papilloma virus (HPV) L1 type 18 polypeptide or fragment thereof comprising at least one peptide comprising an epitope of an L2 polypeptide inserted within the HPV L1 polypeptide. In one embodiment the polypeptide comprises at least two peptides of a L2 polypeptide.

In an alternate embodiment the invention provides a human papilloma virus (HPV) L1 type 16 polypeptide or fragment thereof comprising a peptide comprising amino acids 56-75 of an HPV L2 polypeptide inserted within the HPV L1 polypeptide.

The chimeric polypeptides of the invention can be presented as capsomeres or Virus like particles (VLP). Such polypeptides, capsomeres and VLPs and can be formulated in to immunogenic compositions. Methods of their manufacture, sure and for their use e.g. in the formulation of medicines for the prevention of HPV infections are described.

The invention further provides a composition comprising:

(i) at least one human papillomavirus (HPV) L1 virus like particle (VLP); and

(ii) at least one chimeric HPV L1 polypeptide, capsomere or VLP, comprising an L2 peptide in the L1 sequence.

The invention further provides a composition comprising a combination of two or more chimeric HPV L1 polypeptide, capsomeres or VLPs, each L1 comprising an L2 peptide in the L1 sequence.

The invention further provides a composition comprising:

(i) at least one human papillomavirus (HPV) L1 virus like particle (VLP); and

(ii) at least one chimeric HPV L1 polypeptide, capsomere or VLP comprising an L2 peptide in the L1 sequence, for use in the prevention or treatment of a disorder related to HPV infection.

The invention further provides a composition comprising a combination of two or more chimeric human papillomavirus (HPV) L1 polypeptides, capsomeres or VLPs comprising an L2 peptide in the L1 sequence, for use in the prevention or treatment of a disorder related to HPV infection.

The invention further provides the use of:

(i) at least one human papillomavirus (HPV) L1 virus like particle (VLP); and

(ii) at least one chimeric HPV L1 polypeptide, capsomere or VLP comprising an L2 peptide in the L1 sequence, in the preparation of a medicament for prevention or treatment of a disorder related to HPV infection.

The invention further provides the use of a combination of two or more chimeric HPV

L1 polypeptides, capsomeres or VLPs comprising an L2 peptide in the L1 sequence, in the preparation of a medicament for prevention or treatment of a disorder related to HPV infection.

The invention further provides a chimeric HPV L1 polypeptides, capsomeres or VLPs comprising two or more L2 peptides in the L1 sequence.

In another aspect the invention provides a method for inducing antibodies against HPV in humans comprising administering to a human an immunogenic composition according to the invention described herein.

In another aspect the invention provides a method for inducing neutralising antibodies against HPV in humans comprising administering to a human an immunogenic composition according to the invention described herein. Such a method can also induce cross neutralising antibodies.

In another aspect the invention provides a method for inducing cellular immunity against HPV in humans comprising administering to a human an immunogenic composition according to the invention described herein.

In another aspect the invention provides a method for inducing neutralising antibodies and cellular immunity against HPV in humans comprising administering to a human an immunogenic composition according to the invention described herein. Such a method can also induce cross neutralising antibodies.

The invention further provides a method for preventing HPV infection or HPV disease related to HPV infection, which method comprises administering to a human an immunogenic composition according to the invention.

The invention further provides a method of preparing an immunogenic composition which method comprises combining (i) at least one human papillomavirus (HPV) L1 virus like particle (VLP), with (ii) at least one chimeric HPV L1 polypeptide, capsomere or VLP comprising an L2 peptide in the L1 sequence, and (iii) a pharmaceutically acceptable diluent or carrier and optionally (iv) an adjuvant, to produce an immunogenic composition as described herein. The invention further provides methods for the purification of the chimeric polypeptides as described herein, said method comprising anion exchange chromatography and hydroxapatite chromatography.

The invention further provides a method of preparing an immunogenic composition which method comprises combining two or more chimeric HPV L1 polypeptides, capsomeres or VLPs comprising an L2 peptide in the L1 sequence.

The invention further provides a method of preparing a composition comprising combining an HPV L1 polypeptide which comprises a peptide epitope of an L2 polypeptide inserted within the L1 polypeptide and a pharmaceutically acceptable diluent or carrier.

### BRIEF DESCRIPTION OF THE DRAWINGS

FIG. 1 shows a C terminally truncated L1 sequence for HPV 16 and HPV 18. The amino acid numbering for the HPV 16 and HPV 18 sequences of FIGS. 1(a) and (b) respectively is used throughout the specification in relation to L1 of HPV 16 and HPV 18.

FIG. 2 shows the exposed loops of HPV 16 and HPV 18 μl and exemplary locations for insertion of L2 peptides into the L1 sequence.

FIG. 3 shows alignments for L1 sequences for HPV 16, 18 and other types, in the exposed loop regions and C terminus invading arm region. The sequence at the top is the HPV 16 L1 sequence shown in FIG. 1.

FIG. 4 shows L2 peptides from various different HPV types.

FIG. 5 shows a flow-chart for the purification of chimeric L1/L2 polypeptides.

### DETAILED DESCRIPTION

### Introduction

This disclosure concerns compositions and methods for the prevention and treatment of disease caused by infection with human papillomavirus (HPV). More specifically, this disclosure relates to chimeric polypeptides containing immunogenic components of the major capsid protein, L1, and the minor capsid protein, designated L2. The chimeric polypeptides disclosed herein include an L1 polypeptide into which at least one L2 peptide has been inserted. The L2 peptide is selected to include at least one epitope of an L2 polypeptide.

In an embodiment, L1 polypeptide is an HPV type 18 L1 polypeptide. Thus, the chimeric L1/L2 polypeptide includes HPV type 18 L1 polypeptide or fragment thereof into which is inserted at least one peptide that includes an epitope of an L2 polypeptide. For example, the L2 peptide can be from a type of HPV other than type 18 (that is, a non-HPV type 18 L2 peptide). Favourably, the L1/L2 polypeptide is capable of inducing an immune response to a native protein comprising the L2 polypeptide from which the peptide is selected. Additionally, the L1/L2 polypeptide can be capable of inducing an immune response to at least one additional native L2 protein.

In an embodiment, the L2 peptide(s) is selected from amino acids 1-200 of the N-terminus of an HPV L2 polypeptide, such as from amino acids 1-150 of the N-terminus of an HPV L2 polypeptide. In specific embodiments, the L2 peptides are selected from the group selected of: a peptide comprising amino acid residues 17-36 of an HPV L2 polypeptide; a peptide comprising amino acid residues 56-75 of an HPV L2 polypeptide; a peptide comprising amino acid residues 96-115 of an HPV L2 polypeptide; and a peptide comprising amino acid residues 108-120 of an HPV L2 polypeptide. In various embodiments, the L2 peptide, or peptides, includes an amino acid sequence represented by SEQ ID NOs:1-31. In one exemplary embodiment, the L2 peptide consists of amino acids 17-36 of HPV type 33 L2 (which is identical to amino acids 17-36 of HPV type 11 L2). In another exemplary embodiment, the L2 peptide consists of amino acids 56-75 of HPV type 58 L2 (which is identical to amino acids 56-75 of HPV type 6 L2). More generally, an L2 peptide can be selected to include at least 8 contiguous amino that are identical to the L2 polypeptides of at least two different HPV types (that is a consensus sequence between two or more types of HPV).

In another embodiment, the chimeric L1/L2 polypeptide includes an HPV L1 type 16 polypeptide or fragment thereof into which has been inserted a peptide comprising amino acids 56-75 of an HPV L2 polypeptide. For example, the L2 peptide can include amino acids 56-75 of an HPV L2 polypeptide from an oncogenic type of HPV, such as HPV type 58.

In certain embodiments, the L2 peptide that is inserted into the L1 polypeptide to form a chimeric L1/L2 polypeptide includes at least one amino acid insertion, deletion, or substitution as compared to a native L2 polypeptide. In an embodiment, the L2 peptide has at least one amino acid insertion, deletion or substitution that removes a disulphide bond between two cysteines or removes the amino acids between two cysteines capable of forming a disulphide bond. Favourably, a polypeptide that includes an L2 peptide with an amino acid insertion, deletion or substitution is capable of inducing an immune response to at least one native L2 protein (or naturally occurring L2 polypeptide).

In an embodiment, the HPV L1 protein has a C-terminal deletion of one or more amino acids. In certain embodiments, the L2 peptide(s) are inserted into an exposed region of the L1 polypeptide. In various embodiments, the exposed loop can be the DE loop (e.g., between amino acids 132-142); the FG Loop (e.g., between amino acids 172-182); the HI loop (e.g., between amino acids 345-359); and/or the C terminus of the L1 polypeptide (e.g., between amino acids 429 and 445). In an embodiment, two or more L2 peptides are inserted within the L1 polypeptide. For example, the two or more L2 peptides can be inserted into the same site (e.g., as a contiguous series of amino acids or concatamer), or into different sites, such as into the DE loop and into the C terminus of the L1 polypeptide. Optionally, when inserted into the same site, the two or more L2 peptides can be joined by at least one additional amino acid, such as by a spacer comprising a plurality of amino acids. When two or more L2 peptides are inserted into the L1 polypeptide, the L2 peptides can be the same or different. Typically, the L2 peptide or peptides include at least 8 contiguous amino acids of a native L2 polypeptide.

In certain embodiments, the L2 peptide(s) is inserted within the L1 polypeptide without deleting an amino acid of the L1 polypeptide. In other embodiments, the L2 peptide(s) is inserted into the L1 polypeptide with a deletion of one or more amino acids of the L1 polypeptide.

In some embodiments, the chimeric L1/L2 is in a supramolecular assembly of chimeric polypeptides, for example in polypeptide particles, such as amorphous aggregates, or more ordered structures, e.g. a capsomere or a virus like particle (VLP) or small non VLP structures.

Another aspect of this disclosure pertains to nucleic acid molecules that encode a chimeric L1/L2 polypeptide as described above. Such nucleic acids can be present in a prokaryotic or eukaryotic expression vector. Suitable expression vectors include, for example, recombinant baculovirus. The recombinant nucleic acids, e.g., expression vectors can be introduced (e.g., infected, transfected or transformed) into host cells. Such host cells are also a feature of this disclosure. These host cells can be used to produce the chimeric L1/L2 polypeptides, e.g., by replicating the host cell under conditions suitable for the expression of the recombinant polypeptide. Optionally, the polypeptide can then be isolated and/or purified, e.g., prior to formulation in an immunogenic composition.

Any of the chimeric L1/L2 polypeptides disclosed herein can be used in medicine, e.g., as immunogenic compositions (such as vaccines) for the prevention or treatment of infection or disease caused by HPV. These compositions are suitable for use in methods for inducing antibodies against HPV in humans by administering the immunogenic composition to a human subject. Favourably, administering the immunogenic composition to the human subject induces antibodies that prevent, ameliorate or treat HPV infection or disease.

Thus, the present disclosure also provides immunogenic compositions for use in the prevention, amelioration or treatment of HPV infection or disease. Such immunogenic composition include a chimeric L1/L1 polypeptide (e.g., a protein), capsomere or VLP as described above, in combination with a pharmaceutically acceptable excipient, diluent or carrier. In some embodiments, the immunogenic composition also includes an adjuvant. Suitable adjuvants include an aluminium salt, such as aluminium hydroxide, and/or 3-Deacylated monophoshoryl lipid A (3D-MPL).

In one embodiment, the compositions includes: (i) at least one virus like particle (VLP) comprising or consisting of a human papillomavirus (HPV) L1 polypeptide or fragment thereof; and (ii) at least one chimeric polypeptide comprising a human papillomavirus (HPV) L1 polypeptide or fragment thereof into which has been inserted at least one peptide comprising an epitope of an L2 polypeptide. Any of the chimeric L1/L2 polypeptides disclosed herein (including the aforementioned supramolecular assemblies, polypeptide particles, capsomeres and/or VLPS) is suitable for use in compositions containing VLPs in combination with chimeric L1/L2 polypeptides.

In an embodiment, the VLPs for use in combination with the chimeric L1/L2 polypeptide consist of L1 polypeptides or fragments thereof. In specific embodiments, the HPV L1 VLPs are HPV 16 and/or HPV 18 L1 VLPs. Similarly, the chimeric polypeptides can also include an HPV 16 L1 polypeptide or fragment thereof and/or an HPV 18 L1 polypeptide of fragment thereof.

In an embodiment such a composition in an exemplary embodiment includes at least one chimeric polypeptide of (ii) which consists of an HPV 16 L1 polypeptide or fragment thereof, an HPV 18 L1 polypeptide of fragment thereof, or both an HPV 16 L1 polypeptide or fragment thereof and an HPV 18 L1 polypeptide of fragment into which a L2 peptide has been inserted thereof. As disclosed above, the chimeric peptides can include an L1 polypeptide with a C terminal deletion of one or more amino acids of the L1 polypeptide. In one specific embodiment, the immunogenic composition, includes both HPV 16 L1 VLPs and HPV 18 L1 VLPs, and chimeric polypeptides with both an HPV 16 L1 polypeptide and an HPV 18 L1 polypeptide. In such an embodiment, the chimeric polypeptide with the HPV 16 L1 polypeptide and the chimeric polypeptide with the HPV 18 polypeptide can include different L2 peptides. Exemplary L1 fragments include HPV 16 L1 devoid of the C terminal 34 amino acids or HPV 18 L1 devoid of the C terminal 35 amino acids.

Similarly, in another embodiment, the immunogenic composition can include a combination of two or more chimeric polypeptides that include a human papillomavirus (HPV) L1 polypeptide or fragment thereof with at least one peptide comprising an epitope of an L2 polypeptide inserted within the HPV L1 polypeptide. For example, the combination can include chimeric polypeptides with the same or different L1 component. Similarly, the chimeric polypeptides in the combination can include the same or different L2 components. In one specific embodiment, the chimeric polypeptides comprise L1 polypeptides of the same HPV type and the L2 peptides are different.

In exemplary formulations, the immunogenic compositions include between 10 and 50 μg of each VLP and/or chimeric polypeptide per human dose. In an embodiment, each VLP and/or chimeric polypeptide is present in an amount of approximately 20 μg.

Immunogenic compositions as described herein can be prepared by combining at least one chimeric polypeptide comprising a human papillomavirus (HPV) L1 polypeptide or fragment thereof with at least one inserted peptide comprising an epitope of an L2 polypeptide inserted within the HPV L1 polypeptide, with at least one other chimeric polypeptide, or with at least one human papillomavirus (HPV) L1 virus like particle (VLP), along with a pharmaceutically acceptable diluent or carrier and optionally an adjuvant.

### Terms

In order to facilitate review of the various embodiments of this disclosure, the following explanations of terms are provided. Additional terms and explanations can be provided in the context of this disclosure.

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

Although methods and materials similar or equivalent to those described herein can be used in the practice or testing of this disclosure, suitable methods and materials are described below. The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example.”

The term “human papillomavirus,” abbreviated “HPV” refer to the members of the genus Papillomavirus that are capable of infecting humans. There are two major groups of HPVs (genital and cutaneous groups), each of which contains multiple virus “types” or “strains” (e.g., HPV 16, HPV 18, HPV 33, HPV 58, etc.) categorized by genetic similarity. In the context of this disclosure the term “type” can be used to designate an HPV, and/or a polypeptide from a specified type of HPV. When prefaced by the term “non-,” the designated HPV or polypeptide is at least one other or additional type of HPV than that referenced. For example, “HPV type 18 L1 polypeptide” refers to the L1 polypeptide of HPV type 18. By contrast, “non-HPV type 18 L1 polypeptide” refers to an L1 polypeptide of any type other than HPV type 18.

The term “polypeptide” refers to a polymer in which the monomers are amino acid residues which are joined together through amide bonds. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “fragment,” in reference to a polypeptide, refers to a portion (that is, a subsequence) of a polypeptide. The term “immunogenic fragment” refers to all fragments of a polypeptide that retain at least one predominant immunogenic epitope of the full-length reference protein or polypeptide. Orientation within a polypeptide is generally recited in an N-terminal to C-terminal direction, defined by the orientation of the amino and carboxy moieties of individual amino acids. Polypeptides are translated from the N or amino-terminus towards the C or carboxy-terminus

The terms “polynucleotide” and “nucleic acid sequence” refer to a polymeric form of nucleotides at least 10 bases in length. Nucleotides can be ribonucleotides, deoxyribonucleotides, or modified forms of either nucleotide. The term includes single and double forms of DNA. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. In one embodiment, a polynucleotide encodes a polypeptide. The 5′ and 3′ direction of a nucleic acid is defined by reference to the connectivity of individual nucleotide units, and designated in accordance with the carbon positions of the deoxyribose (or ribose) sugar ring. The informational (coding) content of a polynucleotide sequence is read in a 5′ to 3′ direction.

The term “heterologous” with respect to a nucleic acid, a polypeptide or another cellular component, indicates that the component occurs where it is not normally found in nature and/or that it originates from a different source or species.

The terms “native” and “naturally occurring” refer to an element, such as a protein, polypeptide or nucleic acid, that is present in the same state as it is in nature. That is, the element has not been modified artificially. It will be understood, that in the context of this disclosure, there are numerous native/naturally occurring types of HPV (and HPV proteins and polypeptides), e.g., obtained from different naturally occurring types of HPV.

A “variant” when referring to a nucleic acid or a polypeptide (e.g., an HPV L1 or L2 nucleic acid or polypeptide) is a nucleic acid or a polypeptide that differs from a reference nucleic acid or polypeptide. Usually, the difference(s) between the variant and the reference nucleic acid or polypeptide constitute a proportionally small number of differences as compared to the referent. A variant nucleic acid can differ from the reference nucleic acid to which it is compared by the addition, deletion or substitution of one or more nucleotides, or by the substitution of an artificial nucleotide analogue. Similarly, a variant polypeptide can differ from the reference polypeptide to which it is compared by the addition, deletion or substitution of one or more amino acids, or by the substitution of an amino acid analogue.

An “antigen” is a compound, composition, or substance that can stimulate the production of antibodies and/or a T cell response in an animal, including compositions that are injected, absorbed or otherwise introduced into an animal. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

An “adjuvant” is an agent that enhances the production of an immune response in a non-specific manner. Common adjuvants include suspensions of minerals (alum, aluminum hydroxide, aluminum phosphate) onto which antigen is adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Toll-like Receptor agonists (particularly, TLR2, TLR4, TLR7/8 and TLR9 agonists), and various combinations of such components.

An “immunogenic composition” is a composition of matter suitable for administration to a human or animal subject (e.g., in an experimental setting) that is capable of eliciting a specific immune response, e.g., against a pathogen, such as Human Papillomavirus. As such, an immunogenic composition includes one or more antigens (for example, antigenic subunits of viruses, e.g., polypeptides, thereof) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or treated, e.g., reduced or ameliorated) by inhibiting replication of the pathogen (e.g., Human papillomavirus) following exposure of the subject to the pathogen. For example, in the context of this disclosure, certain embodiments of immunogenic compositions that are intended for administration to a subject or population of subjects for the purpose of eliciting a protective or palliative immune response against human papillomavirus are vaccine compositions or vaccines.

### Chimeric L1/L2 Polypeptides

The present invention is directed towards polypeptides that can be formulated into vaccine compositions, and that satisfy the need for a safe and effective vaccine composition to provide protection against HPV infection and/or disease and which differs from currently available commercial vaccines. In particular, the present invention concerns chimeric polypeptides that include an HPV L1 polypeptide into which has been inserted at least one peptide that includes an antigenic epitope of an HPV L2 polypeptide.

The HPV L1 and L2 polypeptides disclosed herein may be from any genotype of HPV including in particular the high risk cancer causing HPV types HPV 16, 18, 31, 33, 35, 39, 45, 51, 52, 56, 58, 59, 66, 68 or 73 and the genital warts causing HPV types such as HPV 6 or 11 and the skin causing types such as types HPV5 and HPV8 or even types 2 and 3 associated with common warts, and HPV76 associated with benign cutaneous warts.

For example, in an embodiment the present invention provides a human papilloma virus (HPV) L1 type 18 polypeptide or fragment thereof comprising at least one peptide comprising an epitope of an L2 polypeptide inserted within the HPV L1 polypeptide. An epitope of an L2 polypeptide is a peptide that when properly presented is capable of inducing an immune response that will recognise a native (e.g., full length) L2 polypeptide from a human papillomavirus, for example, a naturally occurring human papillomavirus.

In another embodiment there is provided a human papilloma virus (HPV) L1 type 16 polypeptide or fragment thereof comprising at least one peptide comprising amino acids 56-75 from an HPV L2 polypeptide.

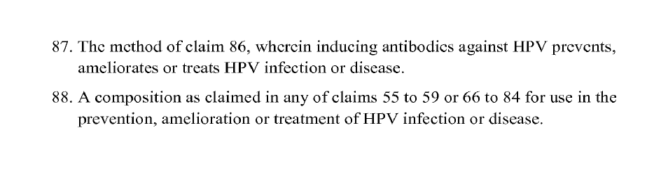

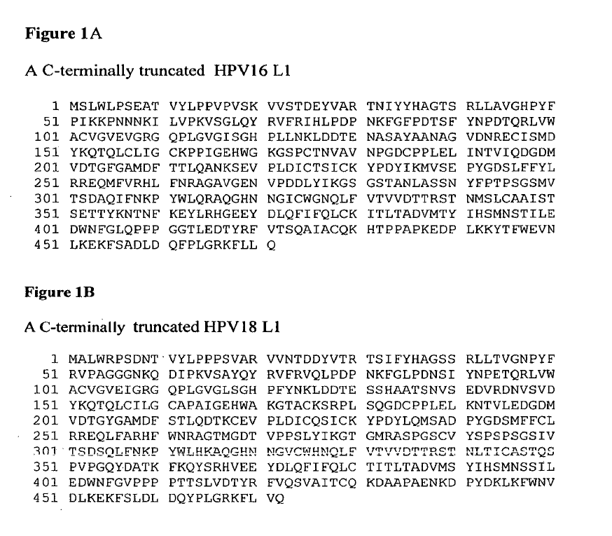

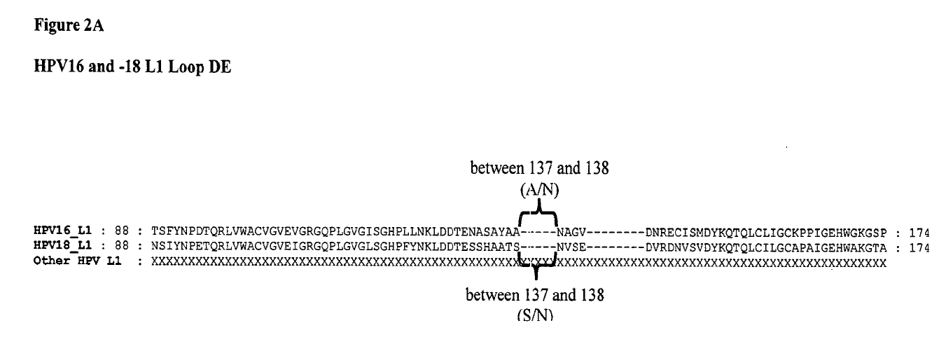

The L1 polypeptide can be a full-length L1 polypeptide. In certain embodiments, the L1 polypeptide is a fragment of L1, such as a fragment truncated by the deletion of one or more amino acids from the N- or C-terminus. Accordingly, in certain embodiments, the L1 polypeptides are truncates from which one or more amino acids are removed from one or both ends compared to the native protein (that is the protein as found in nature). In a particular embodiment, the L1 polypeptide has a C-terminal deletion. An exemplary L1 HPV 16 sequence is given in FIG. 1a. An Exemplary L1 HPV 18 sequence is given in FIG. 1b.

The truncated L1 proteins maybe capable of self-assembly, e.g., into capsomeres or VLPs. Virus like particles typically resemble HPV viruses under the electron microscope. Typically they are made up of 72 capsomeres which in turn are made up of 5 L1 polypeptides in a pentameric unit. Suitably at least one of the L1 proteins utilised herein, comprises a truncated L1 protein, and where multiple HPV VLPs, chimeric polypeptides or capsomeres are present, suitably all the L1 proteins in the composition are truncated L1 proteins. Suitably the truncation removes a nuclear localisation signal. Suitably the truncation is a C-terminal truncation. Suitably the C-terminal truncation removes fewer than 50 amino acids, for example fewer than 40 amino acids. In one particular embodiment the C terminal truncation removes 34 amino acids from HPV 16 and 35 amino acids from HPV 18.

Truncated L1 proteins employed herein are suitably functional L1 protein derivatives or variants. Functional L1 protein derivatives or variants are capable of raising an immune response (optionally, when suitably adjuvanted), said immune response being capable of recognising a virus comprising (or VLP consisting of) the full length L1 protein and/or the HPV type from which the L1 protein was derived.

The location of the L2 peptide in a chimeric HPV L1 polypeptide disclosed herein is important.

In any embodiment disclosed herein the L2 peptide can be located in one of the exposed loops or the C terminus invading arm of the L1 protein. The loops and invading arm are found when the L1 is in the form of capsomers or virus like particles (Chen et al 2000 Mol Cell 5, 557-567).

In any embodiment disclosed herein the L2 peptide can be located at a position selected from the following regions of the L1 sequence, where the locations relate to the HPV 16 and HPV 18 L1 reference sequence shown in FIG. 1, or at an equivalent position in another HPV L1 sequence:

(i) BC loop in amino acids 50-61

(ii) DE loop in amino acids 132-142, for example amino acids 132-141, particularly amino acids 137-138

(iii) EF loop in amino acids 172-182, for example 176-182, particularly 176-179

(iv) FG loop in amino acids 271-290, for example 272-275, particularly 272-273

(v) HI loop in amino acids 345-359, for example 347-350, particularly 349-350

(vi) C terminus arm in amino acids 429-445, for example 423-440, particularly 423-424, 431-433, or 437-438 for HPV 16, and 424-425, 432-433 or 439-440 for HPV 18.

In any embodiment disclosed herein the HPV L2 peptide can be inserted into the L1 sequence without removing L1 amino acids. Alternatively the L2 peptide can be inserted into the L1 sequence with removal of one or more amino acids from the L1 sequence at the position of insertion, for example 1, 2, 3, 4, 5, 6, 7, 8, 9, 10, 11, 12, 13, 14, 15, 16, 17, 18, 19 or 20 amino acids of the L1 sequence can be removed at the location where the L2 peptide is inserted. Thus the L2 peptide can substitute for one or more amino acids in the L1 sequence, for example the L2 peptide can replace an L1 sequence of equivalent length to the L2 peptide sequence.

Where two or more L2 peptides are present in a chimeric L1/L2 polypeptide, these can be different L2 peptides from the same HPV type, or they can be peptides from different HPV types in which case they can be from the corresponding region in the different HPV types.

In an embodiment, the L2 peptide is inserted into a site which permits assembly of a supramolecular assembly of chimeric polypeptides, for example in polypeptide particles, such as capsomers or virus like particlea (VLPs) or small non VLP like structures. For example, to maintain VLP structure, the L2 peptide is inserted into the L1 polypeptide at a site that does not interfere with the sites involved in formation of disulphide bridges that are involved in maintaining inter-capsomere interactions and thus VLP conformation. Such supramolecular structures can be assessed by electron microscopy, for example, as described by Sadeyen et al 2003, Virology 309, 32-40; Slupetzky et al, 2007 Vaccine 25, 2001-2010, Varsani et al 2003, J of Virology 77, 8386-8393, Chen et al 2000, Deschuyteneer M et al 2010, Human Vaccines 6:5, 407-419. Typically, the chimeric VLPs are of a similar or identical size as compared to native VLPs, that is, VLPs in which the L1 protein is full length or truncated, but does not contain an L2 peptide. The chimeric VLPs can be in the range of 50 nm in diameter. In alternate embodiments small non-VLP structures of between 20-35 nm in oliameter are formed.

The site at which the L2 peptide is inserted can allow the presence of conformation dependent neutralising epitopes to be maintained. Neutralising epitopes can be detected by using monoclonal antibodies such as V5, H16. E70 and U4 for HPV 16 (Christensen et al 2001, Carter et al 2003, 2006, Day et al., 2007) and J4 for HPV 16 (Combita et al 2002). Additional neutralising epitopes are known in the art and their presence or absence can be similarly identified using monoclonal antibodies.

However, maintenance of all the L1 neutralising epitopes on the L1 polypeptide may not be necessary, e.g., especially in the compositions described herein that also contain non-chimeric L1 VLPs. Suitable sites for insertion of the L2 peptide expose the L2 peptide at the surface of the L1 polypeptide particularly when presented as a VLP, for example the sites shown in Table 1.

Table 1 & 2 shows the HPV L1 exposed regions (Carter et al 2003, Bishop et al 2007, Chen et al 2000) which can provide suitable insertion sites for the L2 peptide, and the hypervariable regions within those regions. Suitably the L2 peptide is inserted into the C terminus invading arm, or into the DE loop or into the FG loop or into the HI loop. Suitably the L2 peptide is inserted into the hypervariable region of the loop or C terminus arm. The regions shown in Table 1 are for HPV 16; similar regions can be identified in L1 of other HPV types and are defined for HPV 18 L2 in table 2.

TABLE 1
HPV L1 exposed loops for HPV 16

| Position of hypervariable region | |
|---|---|
| within loops in L1 sequence | Loop name |
| AA 50-61 | BC loop |
| AA 132-142 | DE loop |
| AA 172-182 | EF loop (Capsomer bridge: |
| | Cys 175 & Cys 428) |
| AA 271-290 | FG loop |
| AA 345-359 | HI loop |
| AA 429-445 | C terminus (Ct) invading arm |

TABLE 2
HPV 18 L1 exposed loops

| Position of hypervariable region | |
|---|---|
| within loops in L1 sequence | Loop name |
| | BC loop |
| AA 132-142 | DE loop |
| | EF loop (Capsomer bridge: |
| | Cys 175 & Cys 428) |
| AA 271-290 | FG loop |
| AA 345-359 | HI loop |
| AA 429-445 | C terminus (Ct) invading arm |

It can be advantageous in a chimeric L1 polypeptide, capsomere, or VLP described herein to insert an L2 peptide into or in the region of an immunodominant epitope, such as the epitope of HPV 16 recognised by the V5 monoclonal antibody. This can result in the immunodominant epitope losing its immunodominance so that another L1 epitope can become immunodominant, which may in turn result in better cross protection. For example an L2 peptide inserted into the FG loop in the region of the epitope recognised by the V5 antibody may result in the epitope losing its immunodominance and a subdominant epitope becoming immunodominant.

Where two or more L2 peptides are present in separate polypeptides (or capsomeres, or VLPs) in a composition described herein, the L2 peptides can be in the L1 from different HPV types, or in L1 from the same HPV type.

In an embodiment comprising two or more L2 peptides in one polypeptide (or capsomere or VLP), the L2 peptides can be inserted in the same or different sites in the L1 sequence. Where the L2 peptides are inserted at the same site, this can be in the same loop and can be in the same hypervariable region of the same loop. It may be advantageous to have a short stretch of amino acids between the L2 peptides for example 1, 2, 3, 4, 5, 6, 7, 8, 9, or 10 amino acids between the L2 peptides.

The L2 peptides are selected to include at least one antigenic epitope. The selected epitope (as incorporated into an L1 polypeptide) is generally capable of eliciting an immune response to at least one native L2 polypeptide, such as an L2 polypeptide that includes the amino acids present in the selected epitope. Favourably, the chimeric L1/L2 polypeptide is capable of eliciting an immune response against at least one additional native L2 protein.

The L2 peptides for use as described herein can be selected from the following peptides:

a peptide comprising amino acid residues 17-36 of L2;

a peptide comprising amino acid residues 56-75 of L2;

a peptide comprising amino acid residues 96-115 of L2;

a peptide comprising amino acid residues 108-120 of L2.

For example, the L2 peptides for use as described herein can be selected from the following peptides:

a peptide consisting of amino acid residues 17-36 of L2;

a peptide consisting of amino acid residues 56-75 of L2;

a peptide consisting of amino acid residues 96-115 of L2;

a peptide consisting of amino acid residues 108-120 of L2.

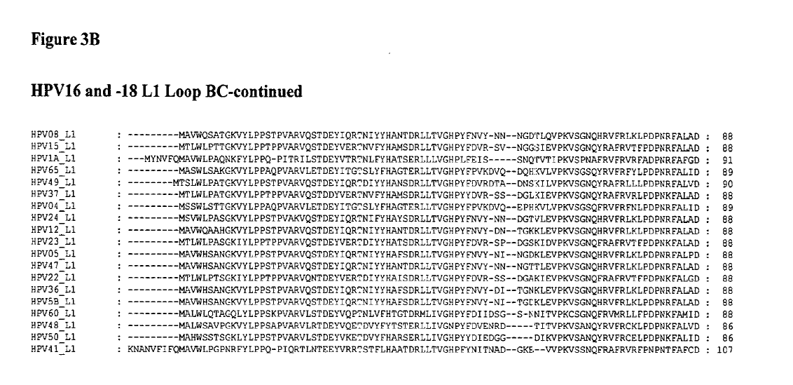

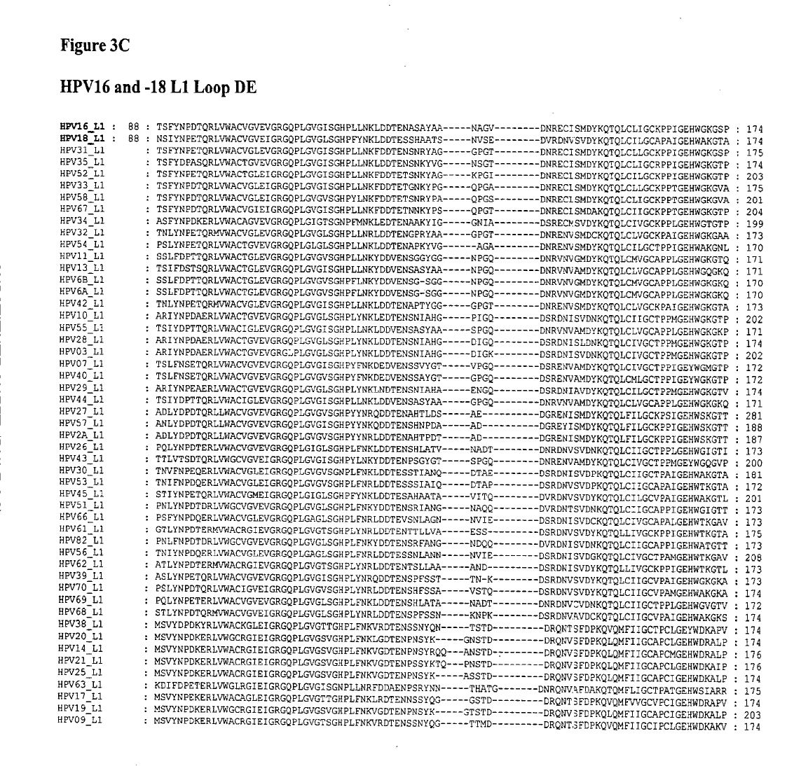

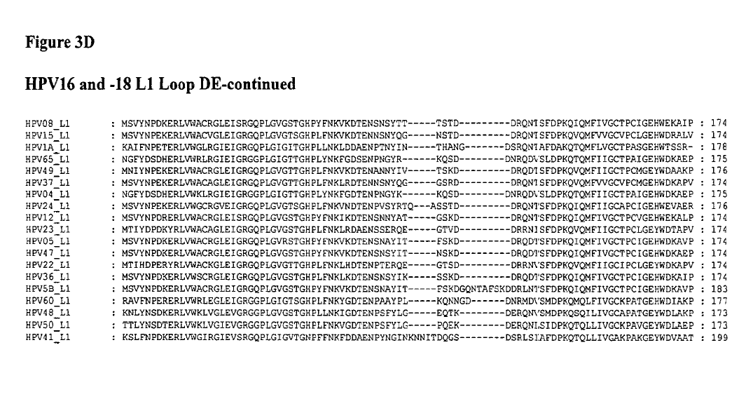

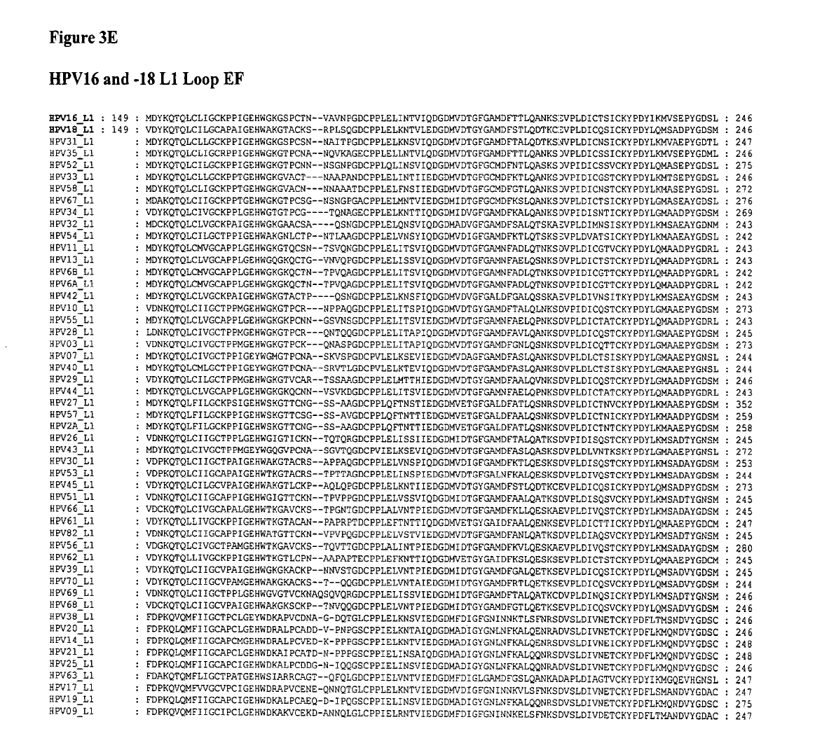

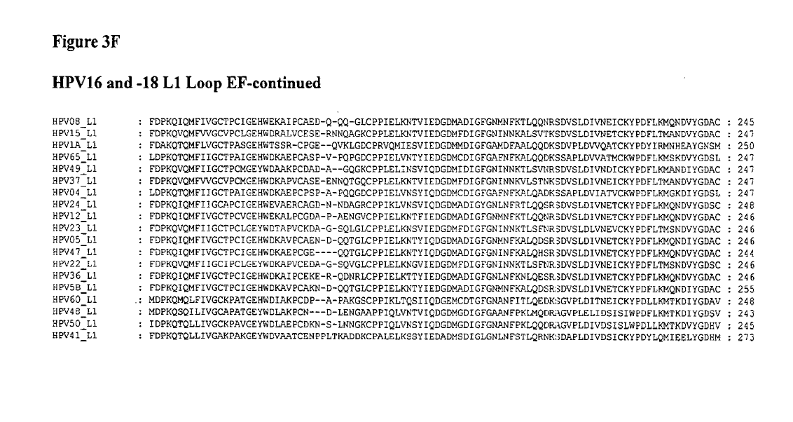

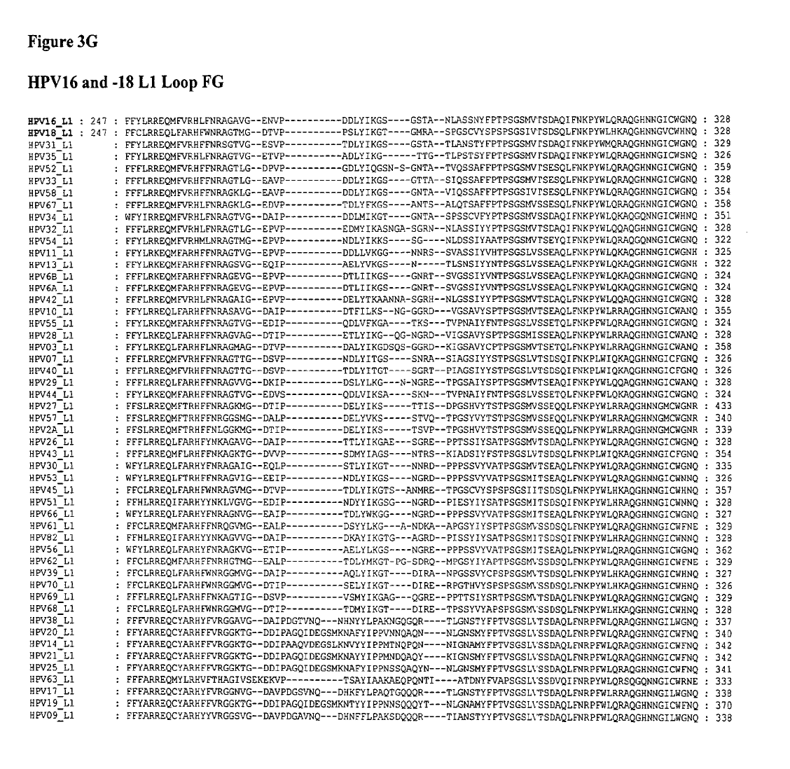

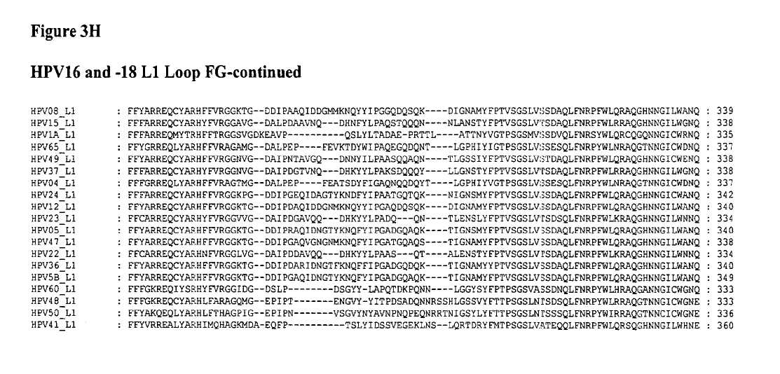

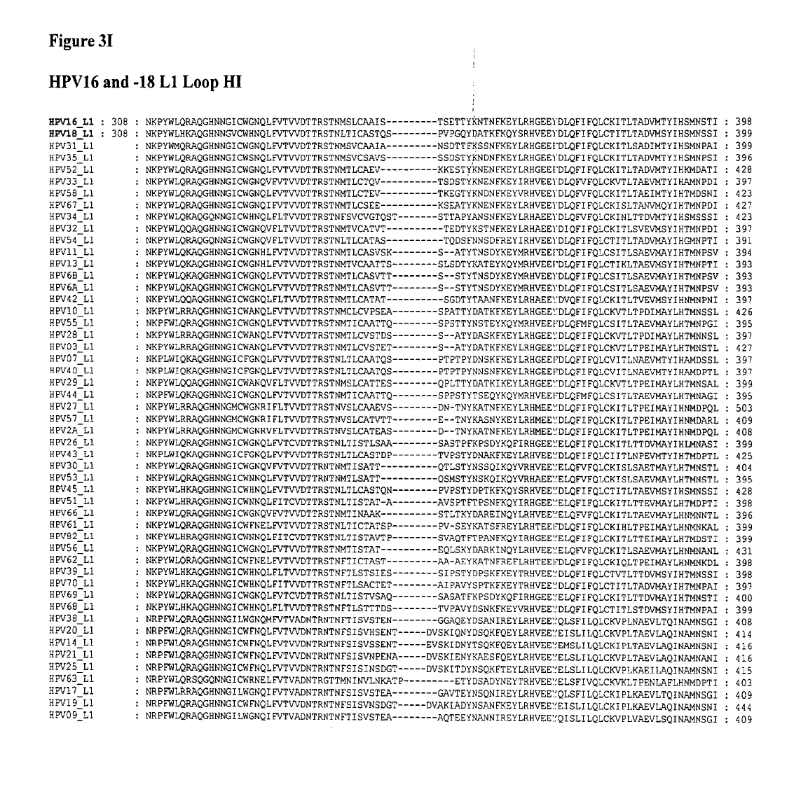

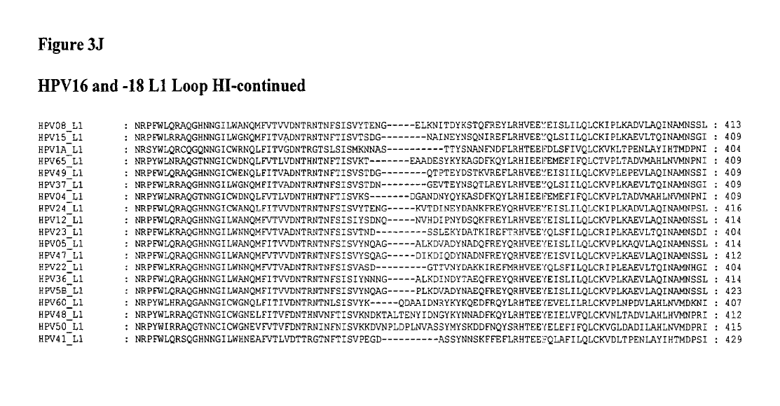

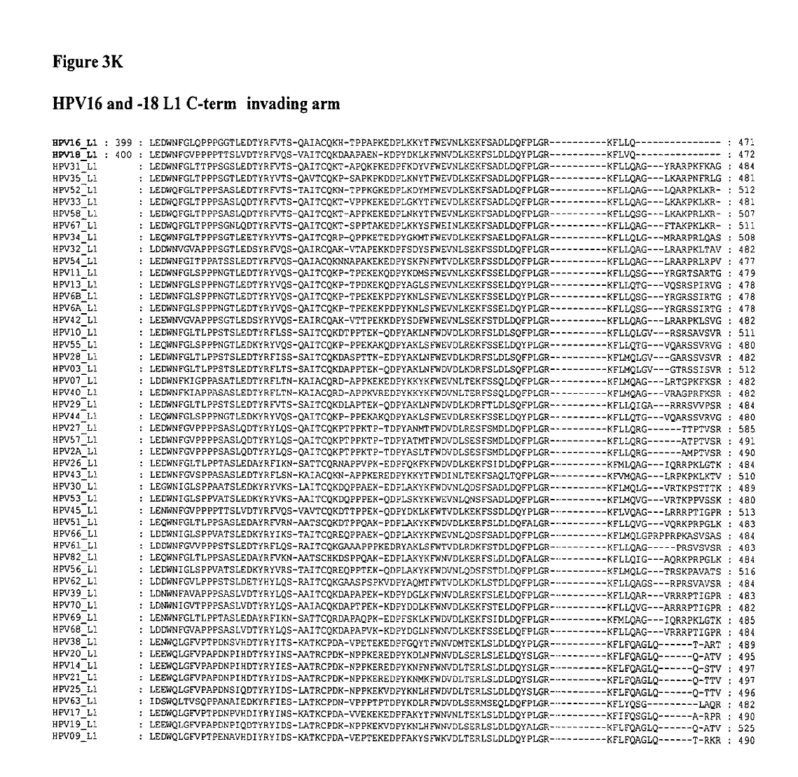

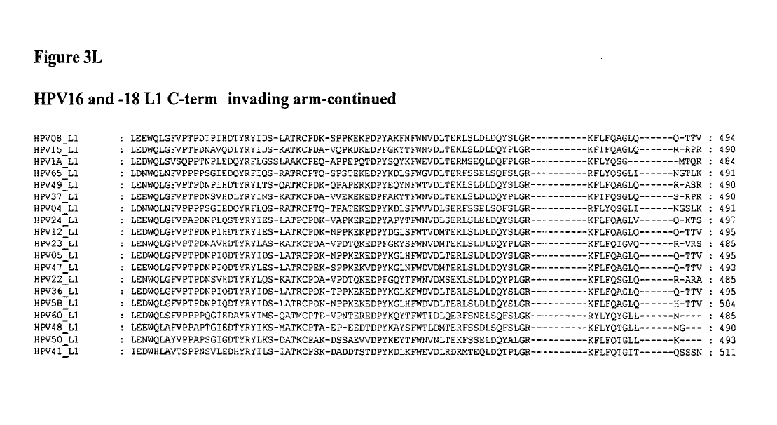

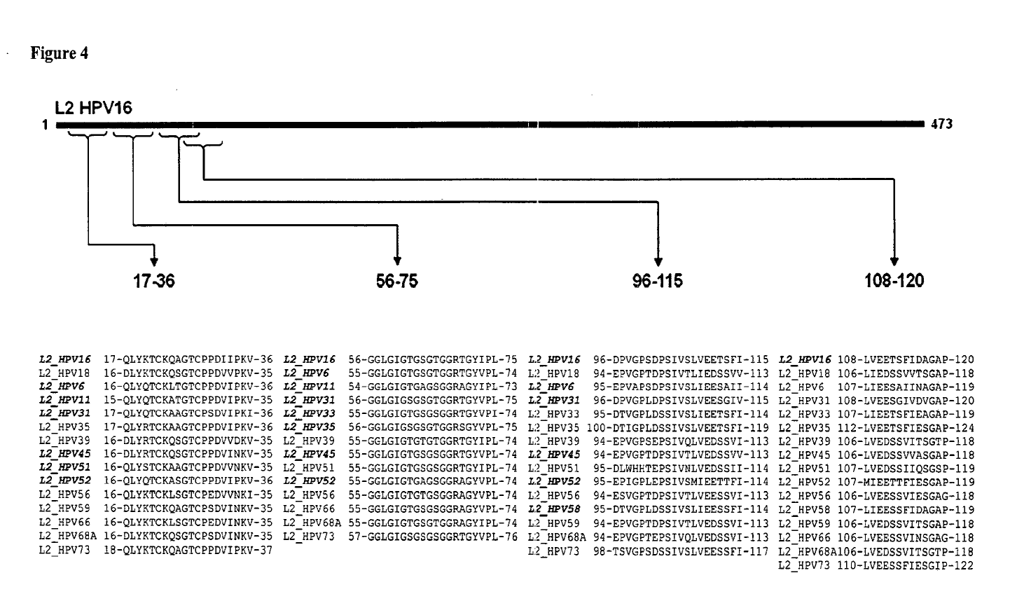

FIG. 3 shows the HPV L2 sequence for HPV16 L2 peptides from positions 17 to 36, 56 to 75, 96 to 115 and 108 to 120 of the L2 amino acid sequence, and for L2 peptides from the corresponding region of various other HPV types. Sequences available in the literature for other known HPV types can be used to design corresponding L2 peptides from additional HPV types according to the HPV 16 L2 sequence as reference sequence (see SwissProt (Boeckmann et al., 2003) or Genbank (Benson et al., 2008)).

Throughout the specification the L2 sequence of HPV 16 is used as the reference sequence to determine the region from which the L2 sequence is derived. Numbering starts at amino acid 1 at the N terminus, with the N terminus at the left hand end of any sequences appearing herein and the C terminus at the right. Actual numbering for certain equivalent peptides from other HPV types is also given, in the lists of specific peptides below.

Suitably the L2 peptide comprises or consists of the L2 56-75 peptide, optionally modified as described herein, for example SEQ ID NO: 8-15, or SEQ ID NO: 29. The L2 56-75 peptide shows substantial sequence identity, i.e., homology, between HPV types.

The L2 peptides according to the present disclosure can comprise or consist of one or more, or two or more of the sequences represented by SEQ ID NOs: 1 to 31.

The L2 peptide(s) can be selected from the segment of amino acids between amino acids 17-36 (e.g., 20 mers), for example:

[SEQ ID NO: 1]
Type 16: 17-QLYKTCKQAGTCPPDIIPKV-36
[SEQ ID NO: 2]
Type 52: 16-QLYQTCKASGTCPPDVIPKV-35
[SEQ ID NO: 3]
Type 51: 16-QLYSTCKAAGTCPPDVVNKV-35
[SEQ ID NO: 4]
Type 6: 16-QLYQTCKLTGTCPPDVIPKV-35
[SEQ ID NO: 5]
Type 11: 15-QLYQTCKATGTCPPDVIPKV-34
[SEQ ID NO: 6]
Type 31: 17-QLYQTCKAAGTCPSDVIPKI-36
[SEQ ID NO: 7]
Type 45: 16-DLYRTCKQSGTCPPDVINKV-35
[SEQ ID NO: 33]
Type 5: 17-HIYQTCKQAGTCPPDVINKV-36
[SEQ ID NO: 34]
Type 56: 17-QLYKTCKLSGTCPEDVVNKI-36

The L2 peptide(s) can also be selected from the segment of amino acids between amino acids 56-75 (e.g., 20 mers), for example:

[SEQ ID NO: 8]
Type 16: 56-GGLGIGTGSGTGGRTGYIPL-75
[SEQ ID NO: 9]
Type 6: 55-GGLGIGTGSGTGGRTGYVPL-74
[SEQ ID NO: 10]
Type 31: 56-GGLGIGSGSGTGGRTGYVPL-75
[SEQ ID NO: 11]
Type 33: 55-GGLGIGTGSGSGGRTGYVPI-74
[SEQ ID NO: 12]
Type 45: 55-GGLGIGTGSGSGGRTGYVPL-74
[SEQ ID NO: 13]
Type 11: 55-GGLGIGTGAGSGGRAGYIPL-74
[SEQ ID NO: 14]
Type 35: 56-GGLGIGSGSGTGGRSGYVPL-75
[SEQ ID NO: 15]
Type 52: 55-GGLGIGTGAGSGGRAGYVPL-74
[SEQ ID NO: 35]
Type 5: 56-GGLGIGTGSGTGGRTGYVPL-75

The L2 peptide(s) can also be shorter than 20 amino acids, for example, the L2 peptides can be any number of amino acids greater than or equal to 8 amino acids, such as 9 amino acids, 10 amino acids, 11 amino acids, 12 amino acids, 15 amino acids, 20 amino acids, or up to 30 amino acids (or an integer of amino acids between 8 and 30). For example, the L2 peptide can be an 8 amino acid segment from between amino acids 56-75, such as the following exemplary 8-mer.

[SEQ ID NO: 29]
Type 16: 56-GGLGIGTG-63

The L2 peptide(s) can also be selected from the segment of amino acids between amino acids 96-115 (e.g., 20 mers), for example:

[SEQ ID NO: 16]
Type 6: 95-EPVAPSDPSIVSLIEESAII-114
[SEQ ID NO: 17]
Type 52: 95-EPIGPLEPSIVSMIEETTFI-114
[SEQ ID NO: 18]
Type 31: 96-DPVGPLDPSIVSLVEESGIV-115
[SEQ ID NO: 19]
Type 16: 96-DPVGPSDPSIVSLVEETSFI-115
[SEQ ID NO: 20]
Type 58: 95-DTVGPLDSSIVSLIEESSFI-114
[SEQ ID NO: 21]
Type 45: 94-EPVGPTDPSIVTLVEDSSVV-113

The L2 peptide(s) can also be selected from the segment of amino acids between amino acids 108-120 (e.g., 13 mers), for example:

[SEQ ID NO: 22]
Type 16: 108-LVEETSFIDAGAP-120
[SEQ ID NO: 23]
Type 18: 106-LIEDSSVVTSGAP-118

Any of the above peptides can be modified, by the addition, deletion or substitution of at least one amino acid, e.g., by the addition, deletion or substitution of one, two or several amino acids. For example in the L2 peptide 17-36 the region (22-28) between the two cysteines and one or both of the cysteines can be deleted. This modification is shown for HPV 16 peptides (SEQ ID NOs. 24 and 25). In another example the valine (V) located four amino acids from the C terminus (i.e., position 32) of HPV type 51 peptide 17-36 can be substituted with an isoleucine (I) which is the amino acid found at this position in all of the other HPV types shown above.

In another example the L2 peptide from 56-75 can be reduced in size (for example, as in SEQ ID NO: 29) provided that the region GGLGI (SEQ ID NO: 32) at the C terminus is maintained because this has been shown to be important for cross reactivity between HPV types (Kondo et al., 2007). Thus an L2 peptide as described herein can comprise the sequence GGGLGI.

Optionally, a spacer of one or more amino acids, such as glycine residues, can also be included at the N or C terminus of the L2 peptide. For example the peptides can further comprise one or two or three added spacer amino acids for example one or two or three amino acid residues added at the amino or the carboxy terminus (or between linked peptides where two or more L2 peptides are present). Generally the spacer will have no specific biological activity other than to join the immunogenic peptide to the L1 sequence, or to preserve some minimum distance or other spatial relationship between them. A spacer may be needed or helpful to retain the correct conformation of the L1 VLP and/or an effective or improved presentation of the inserted L2 peptide compared to absence of a spacer.

Any of the above peptides can be modified, e.g., by the insertion (addition), deletion or substitution of one or more amino acids. For example, the L2 peptides can incorporate amino acids that differ from the L2 sequence of native (that is, naturally occurring) HPV L2 sequence. For example the peptides can have one or two amino acid insertions or substitutions within the sequence, or a deletion of one or two or several amino acids for example 1, 2, 3, 4, 5, 6, 7, 8 or up to 10 amino acids compared to the native sequence for example to remove the occurrence of a disulphide bond between two cysteines and/or the region in between the cysteines. In specific examples, the modifications present in the L2 peptides of the present disclosure, in relation to a native L2 sequence, are limited to 1 or 2 amino acid insertions, deletions, or substitutions, and/or deletion of up to 10 contiguous amino acids between two cysteine residues.

Where modifications to the L2 sequence are made in the peptides described herein, such modification can be limited such that a substantial proportion or at least 50% or at least 70% or at least 90% or at least 95% of the amino acids in the peptide correspond to amino acids in a native L2 sequence.

Alternatively, or additionally, any particular L2 peptide can be a chimera of two or three or more L2 peptides as described herein. In the case of any of these modifications to the L2 sequence, the immunogenic character of the L2 sequence is maintained. That is, the epitope or epitopes of L2 within the peptide which elicits the desired immune response is maintained. The purpose of the modifications can be to improve the properties of the L2 peptide for example to improve cross reactivity with L2 from other HPV types.

Thus the L2 peptides can be selected from the following peptides:

(a) a peptide corresponding to amino acid residues 17-36 of L2 comprising one or more amino acid additions, deletions and/or substitutions;

(b) a peptide corresponding to amino acid residues 56-75 of L2 comprising one or more amino acid additions, deletions and/or substitutions;

(c) a peptide corresponding to amino acid residues 96-115 of L2 comprising one or more amino acid additions, deletions and/or substitutions;

(d) a peptide corresponding to amino acid residues 108-120 of L2 comprising one or more amino acid additions, deletions and/or substitutions; wherein the one or more insertions, deletions and/or substitutions are as compared to a native L2 polypeptide.

The following are examples of L2 peptides with modifications.

[SEQ ID NO: 24]
Type 16: 17-QLYKTCPPDIIPKV-36
(without variable region (23-28) between 2
cysteines and deleting one cysteine)
[SEQ ID NO: 25]
Type 16: 17-QLYKTCPPDVIPKV-36
(without variable region in between 2 cysteines,
deleting one cysteine and substituting Ile with
Val at position 32 (I32V)
[SEQ ID NO: 26]
Type 16: 17-QLYKTCKQAGTCPPDVIPKV-36
(containing I32V)
[SEQ ID NO: 27]
Type 51: 16-QLYSTCKAAGTCPPDVINKV-35
(containing V33I)
[SEQ ID NO: 28]
Type 45: 16-DLYRTCKQSGTCPPDVIPKV-35
(containing N34P)

The L2 peptide can also be a concatamer selected from two different segments of amino acids, e.g., from amino acids 17-36 and amino acids 56-75 (20 mers) for example:

[SEQ ID NO: 30]
Type 16: 17-QLYKTPPDIIPKVGGLGIGTG-63
[SEQ ID NO: 31]
Type 16: 17-QLYKTPPDVIPKVGGLGIGTG-63
(with I32V)

The two peptides represented by SEQ ID NOs: 30 and 31 are chimeras of two of the above peptides and contain the region from peptide 17-36 without both of the cysteines and without the region (22-28) between the cysteines, together with the region 56-63 (conserved between HPVs) from the 56-75 peptide. A similar peptide can be constructed from other HPV types.

It will be evident that any of the above listed peptides can be included in an HPV L1, polypeptides, capsomers, or VLP as described herein, at any of the sites in the L1 sequence as discussed herein.

Where a plurality of L2 peptides are present in a composition according to the present disclosure, these can be two or more different L2 peptides from the same HPV type, i.e. peptides from different (including overlapping) regions of L2, or it can be two or more different L2 peptides from the same region of different HPV types e.g. the 17-36 region. Where more than two L2 peptides are present this can involve both multiple peptides from the same HPV type and multiple peptides from different HPV types.

Where two or more different L2 peptides are present in a composition according to the present disclosure, each peptide can be present in an HPV L1 VLP from a different HPV type for example HPV 16 and HPV 18 L1 VLPs, or they may be in HPV L1 VLPs from the same HPV type for example HPV 16 or HPV 18 L1 VLPs. Suitably L2 peptides described herein are present in HPV VLPs from HPV 16 and/or HPV 18.

Suitably, in any embodiment disclosed herein, the L2 peptides include at least 8 contiguous amino acid residues from the L2 protein. In any embodiment disclosed herein the HPV L2 peptide or peptides can be up to 30 amino acid residues in length.

In any embodiment disclosed herein, L2 peptides can be selected from amino acids 1-200 of the N terminus of HPV L2, particularly amino acids 1-150 of HPV L2. Thus the L2 peptides can comprise 8 or more amino acid residues from the region 1-200 or 1-150 of HPV L2, which can be 8 or more contiguous amino acid residues from the region 1-200 or 1-150.

The term ‘L2 peptide can comprise at least 8 amino acid residues’, as used herein, refers to peptides of any 8 or more amino acids derived from L2, although peptides are suitably at least 9, 10, 11, 12, 13, 14, 15, 20 or more amino acids in length. In one embodiment the L2 peptides of the present disclosure are short peptides of less than 100 amino acids, suitably less than 50 amino acids, or less than 40 amino acids. For example the peptides can be up to 30 amino acids in length, or up to 20 or 21 amino acids in length. The full length L2 protein is not considered to be a peptide of L2 in the context of the chimeric L1/L2 polypeptides disclosed herein.

The minimum requirement for an L2 peptide in the present disclosure is a peptide that is capable of inducing an immune response to a native L2 protein. Thus, the L2 peptides typically include at least 8 contiguous amino acids of an L2 polypeptide, and include at least one epitope. One or more of the different L2 peptides in a composition according to the present disclosure can be up to 25 amino acids or up to 30 or up to 40 amino acids in length. In one embodiment, all of the different L2 peptides used in a composition according to the present disclosure are up to 25 amino acids or up to 30 or up to 40 amino acids in length.

L2 peptides according to the present disclosure are suitably able to elicit an immune response against homologous HPV infection, that is, against infection by the HPV type from which the sequence originates.

Suitably the L2 peptide is capable of inducing an immune response against at least two different HPV types. HPVs are classified by type based on nucleic acid similarity. Numerous HPV types have been described in the literature. Thus, in the context of the present disclosure, an L2 peptide is capable of eliciting an immune response against a specified HPV type, e.g., a particular referenced HPV type, such as HPV type 18 (or any other referenced HPV type). In addition, the L2 peptide, as presented in the context of a chimeric L1/L2 polypeptide disclosed herein, is also capable of eliciting an immune response against an HPV type other than the referenced HPV type. For ease of reference, the HPV type other than the referenced HPV type is referred to as a “non-HPV type.” For example, when the referenced HPV type is HPV 18, any other type than HPV 18 can be referred to as a non-HPV type 18 peptide (or polypeptide or virus). Similarly, with respect to any referenced HPV type, any other type than the referenced HPV type can be referred to as the non-HPV type.

For example, the L2 peptide can induce an immune response against one or more further L2 proteins from different HPV types. In any embodiment disclosed herein the HPV L2 peptide or peptides can be capable of inducing a cross reactive, cross neutralising and/or cross protective response against another HPV type. Suitably the L2 peptide is selected which shows a high level of sequence identity (“homology”) between HPV types that is greater than 80% between two (or more) types. In some cases, the L2 peptide has greater than 85% sequence identity between types, or greater than 90% sequence identity between types, or greater than 95% sequence identity between types. In certain embodiments, the L2 peptide is selected to have 100% sequence identity between at least two HPV types. Such L2 peptides may be referred to herein as L2 “consensus” sequences.

For example, in a particular embodiment, the L2 peptide is a consensus sequence that is identical (i.e., has 100% sequence identity) between HPV type 33 and HPV type 11. For example, in a specific exemplary embodiment, the consensus sequence is identical between amino acids 17-36 of L2. In another embodiment, the L2 peptide is a consensus sequence that is identical between HPV type 58 and HPV type 6. For example, in a specific exemplary embodiment, the consensus sequence is identical between amino acids 56-75 of HPV type 58 and HPV type 6.

Cross reactive L2 peptides which are capable of eliciting an immune response against further HPV types can be identified according to the present disclosure. As shown herein, L2 sequences from different HPV types can be aligned to identify regions with high similarity between HPV types (see FIG. 3 and other sequences shown herein). Numerous sequence programs are available to perform such alignments and identify where there is sequence homology. This can enable selection of L2 peptides which are most similar among HPV types of interest and are therefore potentially cross reactive between some or all of those HPV types.

The L2 peptides of the present disclosure can be any suitable immunogenic L2 peptides. L2 peptides can be tested for immunogenicity and cross reactivity by standard techniques well known in the art. For example, the peptides or chimeric L1 polypeptides, capsomeres or VLPs containing the peptides may be injected into model animals or humans and measurement of antibody and/or cellular immune responses can be carried out for example by ELISA or cytokine analysis/measurement respectively. Methods for screening antibodies are well known in the art. An ELISA can be used to assess cross reactivity of antibodies. Antibodies can be tested for neutralisation and cross neutralisation properties using a pseudovirus neutralisation assay for example. Suitable pseudovirus neutralisation assays are described in Dessy et al 2008 and Pastrana et al 2004.

In addition a number of cross reactive L2 peptides have already been identified. For example a common-neutralising epitope for HPV 6 and 16 has been found in the region (aa) 108-120 of HPV 16 L2 (Kawana et al 1998, 1999, 2001). In another example, immunization of rabbits with (aa) 17-36, 56-75, 96-115 L2 peptides from HPV 16 could give rise to cross neutralising antibodies (Kondo et al 2007, 2008). In another example (aa) 17-36 of L2 from HPV 16 was identified as a protective and broadly cross-neutralising epitope after passive immunization and challenge in BALB/c mice (Gambhira et al 2007). Similar protection was shown in BALB/C mice by Alphs et al 2008 following vaccination with a synthetic lipopeptide vaccine containing aa 17-36 of L2 from HPV 16.

Suitably the L2 peptide or peptides are cross reactive peptides, so that they are able to elicit an immune response which recognises not only the L2 of the HPV genotype from which the L2 peptide is derived, but also an L2 protein or L2 peptide from an HPV genotype other than the one from which it is derived. Suitably the peptide is cross-reactive with 1 or 2 or more other genotypes, suitably a genotype associated with causation of cervical cancer such as HPV type 16, 18, 31, 33, 35, 39, 45, 51, 52, 56, 58, 59, 66, 68 or 73 or genital warts such as HPV 6 or 11, or skin cancer such as HPV 5, 8 or 38.

In one embodiment, one or more of the L2 peptides is selected from an HPV type 16 or a modified version thereof, and is cross reactive against at least one other cancer causing HPV type, such as a type selected from HPV 18, 31, 33, 35, 39, 45, 51, 52, 56, 58, 59, 66, 68 or 73 and/or at least one genital warts causing HPV type such as HPV 6 or 11 and/or at least one skin cancer causing HPV type such as HPV 5, 8 or 38. In another embodiment, one or more of the L2 peptides is selected from and HPV type 18 type or a modified version thereof.

Suitably the L2 peptides used in the invention are capable of generating a cross neutralising immune response, that is an immune response which is capable of neutralising HPV of a different HPV type than the HPV type from which the L2 peptide is derived. Cross neutralisation can be tested for by assays known in the art such as pseudoneutralisation assay described herein in Example 3.

Suitably, the L2 peptide is able to provide cross protection, and suitably comprises a cross neutralising epitope, suitably for one or more of HPV types associated with cervical cancer selected from HPV 16, 18, 31, 33, 35, 39, 45, 51, 52, 56, 58, 59, 66, 68 or 73 and/or at least one genital warts causing HPV type such as HPV 6 or 11 and/or at least one skin cancer causing HPV type such as HPV 5, 8 or 38.

Cross protection suitably occurs when an L2 peptide is capable of generating a protective immune response against infection/disease caused by at least two HPV types. For example, when presented in the context of a chimeric L1/L2 polypeptide, the L2 can induce a response that protects against the type from which the L2 peptide is obtained, and at least one additional type of HPV. As discussed above, cross protection can also occur when a consensus L2 peptide is selected and presented in the context of a chimeric L1/L2 polypeptide. Cross protection against different HPV types different to the one from which the L2 peptide or L1 VLP is derived, can be identified using an animal model, for example a mouse model as described in Alphs et al 2008.

Cross protection can be assessed by comparing incidence of infection and/or disease for a group of HPV types (infection being incident or persistent infection) in individuals vaccinated with a given L2 peptide compared to a non vaccinated group. Complete cross protection against a type, or group of types, is not required according to the present disclosure; indeed, any level of cross protection provides a benefit. Suitably the level of cross protection observed is such that the vaccinated group has 5% less infection and/or disease associated with a non-vaccine HPV type or types, than a comparable non vaccinated group, more suitably up to 10%, up to 15%, up to 20%, up to 25%, up to 30%, up to 35%, up to 40%, up to 45%, up to 50%, up to 55%, up to 60%, up to 65% up to 70%, up to 80%, up to 90% or even up to 100% less infection and/or disease.

Cross protection can be assessed by detecting the presence of nucleic acid specific for various HPV types in the vaccinees and control group. Detection can be carried out, for example, using techniques as described in WO03/014402 (US2007031828A1), and references therein, particularly for non-specific amplification of HPV DNA and subsequent detection of DNA types using a LiPA system as described in WO 99/14377 (U.S. Pat. No. 6,482,588B1), and in Kleter et al, (Journal of Clinical Microbiology (1999), 37 (8): 2508-2517), the whole contents of which are herein incorporated by reference. Any suitable method can, however, be used for the detection of HPV DNA in a sample, such as type specific PCR using primers specific for each HPV type of interest. Suitable primers are known to the skilled person, or can be easily constructed given that the sequences of the different HPV types are known.

Suitably cross protection is observed in the male and/or female population, suitably women who are seronegative for HPV infection, or seronegative for HPV 16 and 18, suitably adolescent women pre-sexual activity.

Cross protection (as assessed by protection seen in a vaccinated group vs. a control group) is suitably seen against oncogenic types, such as any one of the group of high risk cancer types 16, 18, 31, 33, 35, 39, 45, 51, 52, 56, 58, 59, 66, 68 or 73 or, collectively, groups of high risk cancer types such as any 2, 3, 4, 5, 6, 7, 8, 9, 10, 11, 12, 13, 14, or indeed all, of these high risk cancer types. All possible combinations of 2, 3, 4, 5, 6, 7, 8, 9, 10, 11, 12, 13 and 14 of these high risk cancer types are specifically contemplated.

### Nucleic Acids Encoding L1/L2 Polypeptides

Another feature of this disclosure is nucleic acid molecules that encode any of the aforementioned chimeric L1/L2 polypeptides.

Such nucleic acids can be “recombinant” nucleic acids, which have a sequence that is not naturally occurring or has a sequence that is made by an artificial combination of two otherwise separated segments of sequence. For example, the recombinant chimeric L1/L2 nucleic acids include at least one nucleic acid sequence that encodes an HPV L2 peptide operably linked to at least one (and frequently at least two) nucleic acid segments that encode an HPV L1 polypeptide (or fragments thereof). This artificial combination can be accomplished by chemical synthesis or, more commonly, by the artificial manipulation of isolated segments of nucleic acids, e.g., by genetic engineering techniques. For consistentcy, a “recombinant” protein is one that is encoded by a recombinant nucleic acid, (and which may be introduced into a host cell, such as a bacterial or eukaryotic cell).

In certain embodiments, the recombinant nucleic acids that encode chimeric L1/L2 polypeptides are codon optimized for expression in a selected prokaryotic or eukaryotic host cell.

To facilitate replication and expression, the nucleic acids that encode the chimeric L1/L2 polypeptides can be incorporated into a vector, such as a prokaryotic or a eukaryotic expression vector. Host cells including nucleic acids that encode a chimeric L1/L2 polypeptide are also a feature of this disclosure. Favorable host cells include prokaryotic (i.e., bacterial) host cells, such as E. coli, as well as numerous eukaryotic host cells, including fungal (e.g., yeast) cells, insect cells, and mammalian cells (such as CHO, VERO and HEK293 cells).

To facilitate replication and expression, the nucleic acids can be incorporated into a vector, such as a prokaryotic or a eukaryotic expression vector. Although the nucleic acids disclosed herein can be included in any one of a variety of vectors (including, for example, bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others), most commonly the vector will be an expression vector suitable for generating polypeptide expression products. In an expression vector, the nucleic acid encoding the chimeric L1/L2 polypeptide is typically arranged in proximity and orientation to an appropriate transcription control sequence (promoter, and optionally, one or more enhancers) to direct mRNA synthesis. That is, the polynucleotide sequence of interest is operably linked to an appropriate transcription control sequence. Examples of such promoters include: the immediate early promoter of CMV, LTR or SV40 promoter, polyhedrin promoter of baculovirus, E. coli lac or trp promoter, phage T7 and lambda P_{L} promoter, and other promoters known to control expression of genes in prokaryotic or eukaryotic cells or their viruses. The expression vector typically also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as kanamycin, tetracycline or ampicillin resistance in E. coli.

The expression vector can also include additional expression elements, for example, to improve the efficiency of translation. These signals can include, e.g., an ATG initiation codon and adjacent sequences. In some cases, for example, a translation initiation codon and associated sequence elements are inserted into the appropriate expression vector simultaneously with the polynucleotide sequence of interest (e.g., a native start codon). In such cases, additional translational control signals are not required. However, in cases where only a polypeptide-coding sequence, or a portion thereof, is inserted, exogenous translational control signals, including an ATG initiation codon is provided for translation of the nucleic acid encoding the chimeric L1/L2 polypeptide. The initiation codon is placed in the correct reading frame to ensure translation of the polynucleotide sequence of interest. Exogenous transcriptional elements and initiation codons can be of various origins, both natural and synthetic. If desired, the efficiency of expression can be further increased by the inclusion of enhancers appropriate to the cell system in use (Scharf et al. (1994) Results Probl Cell Differ 20:125-62; Bitter et al. (1987) Methods in Enzymol 153:516-544).

In some instances, the nucleic acid (such as a vector) that encodes the chimeric L1/L2 polypeptide includes one or more additional sequence elements selected to increase and/or optimize expression of the encoded polypeptide when introduced into a host cell. For example, in certain embodiments, the nucleic acids that encode the chimeric L1/L2 polypeptide include an intron sequence, such as a Human Herpesvirus 5 intron sequence (see, e.g., SEQ ID NO:13). Introns have been repeatedly demonstrated to enhance expression of homologous and heterologous nucleic acids when appropriately positioned in a recombinant construct.

Exemplary procedures sufficient to guide one of ordinary skill in the art through the production of recombinant nucleic acids that encode chimeric L1/L2 polypeptides can be found in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

Exemplary nucleic acid sequences that encode HPV L1 and L2 polypeptides from the different HPV types are well known in the art, e.g., numerous examples have been described in the literature and are available publicly in the GenBank database. These can readily be identified by those of skill in the art by an appropriate query using the terms human papillomavirus (or HPV) and the specific protein (e.g., L1 or L2) and type of interest. These L1 and L2 nucleic acids can be utilized to produce nucleic acids that encode recombinant chimeric L1/L2 polypeptides as disclosed above.

Additional nucleic acids that encode chimeric L1/L2 nucleic variants that share sequence identity with the exemplary L1 and L2 polypeptide can be produced by those of skill in the art. Typically, the nucleic acid variants will encode polypeptides that differ by no more than 1%, or 2%, or 5%, or 10%, or 15%, or 20% of the amino acid residues present in a chimeric L1/L2 polypeptide (e.g., in the L1 polypeptide portion). That is, the encoded polypeptides share at least 80%, or 85%, more commonly, at least about 90% or more, such as 95%, or even 98% or 99% sequence identity with the reference chimeric polypeptide. It will be immediately understood by those of skill in the art, that the polynucleotide sequences encoding chimeric L1/L2 polypeptides, can themselves share less sequence identity due to the redundancy of the genetic code. In some instances, the encoded L1/L2 polypeptide has one or more amino acid modification relative to the amino acid sequence of the naturally occurring polypeptides from which it is derived. Such differences can result in the addition, deletion or substitution of one or more amino acids. A variant typically differs by no more than about 1%, or 2%, or 5%, or 10%, or 15%, or 20% or of the nucleotide residues. For example, a nucleic acid that encodes a variant chimeric L1/L2 polypeptide can include 1, or 2, or up to 5, or up to about 10, or up to about 15, or up to about 50, or up to about 100 nucleotide differences (e.g., in the L1 portion, and/or to encode the modified L2 peptides as described above). Thus, a variant in the context of a nucleic acid that encodes a chimeric L1/L2 polypeptide as disclosed herein, typically shares at least 80%, or 85%, more commonly, at least about 90% or more, such as 95%, or even 98% or 99% sequence identity with a reference sequence consisting of naturally occurring L1 and L2 components.

In addition to the variant nucleic acids previously described, nucleic acids that hybridize to one or more nucleic acids that encode chimeric L1/L2 polypeptides with L1 and L2 sequences corresponding to naturally occurring L1 and L2 polypeptidescan also be used to encode chimeric L1/L2 polypeptides. One of skill in the art will appreciate that in addition to the % sequence identity measure discussed above, another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993. and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” can be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast, nucleic acids that hybridize under “low stringency conditions include those with much less sequence identity, or with sequence identity over only short subsequences of the nucleic acid.

### Methods for Producing L1/L2 Polypeptides

The chimeric L1/L2 polypeptides disclosed herein can be produced using well established procedures for the expression and purification of recombinant proteins. Procedures sufficient to guide one of skill in the art can be found in the following references: Sambrook et al., Molecular Cloning: A Laboratory Manual, Cold Spring Harbor Laboratory Press, Cold Spring Harbor, N.Y., 200; and Ausubel et al. Short Protocols in Molecular Biology, 4^{th} ed., John Wiley & Sons, Inc., 999. Additional and specific details are provided hereinbelow.

Recombinant nucleic acids that encode the chimeric L1/L2 polypeptides are introduced into host cells by any of a variety of well-known procedures, such as electroporation, liposome mediated transfection (e.g., using a commercially available liposomal transfection reagent, such as LIPOFECTAMINE™2000 or TRANSFECTIN™), Calcium phosphate precipitation, infection, transfection and the like, depending on the selection of vectors and host cells.

Host cells that include chimeric L1/L2 polypeptides-encoding nucleic acids are, thus, also a feature of this disclosure. Favorable host cells include prokaryotic (i.e., bacterial) host cells, such as E. coli, as well as numerous eukaryotic host cells, including fungal (e.g., yeast, such as Saccharomyces cerevisiae and Picchia pastoris) cells, insect cells, plant cells, and mammalian cells (such as CHO and HEK293 cells). Recombinant nucleic acids that encode chimeric L1/L2 polypeptides are introduced (e.g., transduced, transformed or transfected) into host cells, for example, via a vector, such as an expression vector. As described above, the vector can be a plasmid, a viral particle, a phage, a baculovirus, etc. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Trichoplusia, Drosophila, Spodoptera frugiperda; mammalian cells such as 3T3, COS, CHO, BHK, HEK 293 or Bowes melanoma; plant cells, including algae cells, etc.

The host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein. Expression products corresponding to the nucleic acids of the invention can also be produced in non-animal cells such as plants, yeast, fungi, bacteria and the like. In addition to Sambrook, Berger and Ausubel, details regarding cell culture can be found in Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

In bacterial systems, a number of expression vectors can be selected depending upon the use intended for the expressed product. For example, when large quantities of a polypeptide or fragments thereof are needed for the production of antibodies, vectors which direct high level expression of chimeric L1/L2 polypeptides that are readily purified are favorably employed. Such vectors include, but are not limited to, multifunctional E. coli cloning and expression vectors such as BLUESCRIPT (Stratagene), in which the coding sequence of interest, e.g., a polynucleotide of the invention as described above, can be ligated into the vector in-frame with sequences for the amino-terminal translation initiating Methionine and the subsequent 7 residues of beta-galactosidase producing a catalytically active beta galactosidase fusion protein; pIN vectors (Van Heeke & Schuster (1989) J Biol Chem 264:5503-5509); pET vectors (Novagen, Madison Wis.), in which the amino-terminal methionine is ligated in frame with a histidine tag; and the like.

Similarly, in yeast, such as Saccharomyces cerevisiae, a number of vectors containing constitutive or inducible promoters such as alpha factor, alcohol oxidase and PGH can be used for production of the desired expression products. For reviews, see Berger, Ausubel, and, e.g., Grant et al. (1987; Methods in Enzymology 153:516-544). In mammalian host cells, a number of expression systems, including both plasmis and viral-based systems, can be utilized.

A host cell is optionally chosen for its ability to modulate the expression of the inserted sequences or to process the expressed protein in the desired fashion. Such modifications of the protein include, but are not limited to, glycosylation, (as well as, e.g., acetylation, carboxylation, phosphorylation, lipidation and acylation). Different host cells such as 3T3, COS, CHO, HeLa, BHK, MDCK, 293, WI38, etc. have specific cellular machinery and characteristic mechanisms for such post-translational activities and can be chosen to ensure the correct modification and processing of the introduced, foreign protein.

In certain examples, the nucleic acids are introduced into cells via vectors suitable for introduction and expression in prokaryotic cells, e.g., E. coli cells. The expression vector is introduced (e.g., by electroporation) into a suitable bacterial host. Numerous suitable strains of E. coli are available and can be selected by one of skill in the art (for example, the Rosetta and BL21 (DE3) strains have proven favorable for expression of recombinant vectors containing polynucleotide sequences that encode a chimeric L1/L2 polypeptide.

More typically, the polynucleotides that encode the chimeric L1/L2 polypeptide are incorporated into expression vectors that are suitable for introduction and expression in eukaryotic (e.g., insect or mammalian cells). Favorably, such nucleic acids are codon optimized for expression in the selected vector/host cell.

In one example, the polynucleotide sequence that encodes the chimeric L1/L2 polypeptide is introduced into insect cells using a Baculovirus Expression Vector System (BEVS). Recombinant baculovirus capable of infecting insect cells can be generated using commercially available vectors, kits and/or systems, such as the BD BaculoGold system from BD BioScience. Briefly, a polynucleotide sequence encoding the chimeric L1/L2 polypeptide is inserted into the pAcSG2 transfer vector. Then, host cells SF9 (Spodoptera frugiperda) are co-transfected by pAcSG2-chimeric plasmid and BD BaculoGold, containing the linearized genomic DNA of the baculovirus Autographa califormica nuclear polyhedrosis virus (AcNPV). Following transfection, homologous recombination occurs between the pACSG2 plasmid and the Baculovirus genome to generate the recombinant virus. In one example, the chimeric L1/L2 polypeptide antigen is expressed under the regulatory control of the polyhedrin promoter (pH). Similar transfer vectors can be produced using other promoters, such as the basic (Ba) and p10 promoters. Similarly, alternative insect cells can be employed, such as SF21 which is closely related to the Sf9, and the High Five cell line derived from a cabbage looper, Trichoplusia ni.

For long-term, high-yield production of recombinant chimeric L1/L2 polypeptides disclosed herein, stable expression systems are typically used. For example, cell lines which stably express a chimeric L1/L2 polypeptide are introduced into the host cell using expression vectors which contain viral origins of replication or endogenous expression elements and a selectable marker gene. Following the introduction of the vector, cells are allowed to grow for 1-2 days in an enriched media before they are switched to selective media. The purpose of the selectable marker is to confer resistance to selection, and its presence allows growth and recovery of cells which successfully express the introduced sequences. For example, resistant groups or colonies of stably transformed cells can be proliferated using tissue culture techniques appropriate to the cell type. Host cells transformed with a nucleic acid encoding a chimeric L1/L2 polypeptide are optionally cultured under conditions suitable for the expression and recovery of the encoded protein from cell culture.

Following transduction of a suitable host cell line and growth of the host cells to an appropriate cell density, the selected promoter is induced by appropriate means (e.g., temperature shift or chemical induction) and cells are cultured for an additional period.

The secreted polypeptide product is then recovered and/or purified from the culture medium. The term “purification” (e.g., with respect to a chimeric L1/L2 polypeptide, or nucleic acid encoding such a polypeptide) refers to the process of removing components from a composition, the presence of which is not desired. Purification is a relative term, and does not require that all traces of the undesirable component be removed from the composition. In the context of protein production, purification includes such processes as centrifugation, dialization, ion-exchange chromatography, and size-exclusion chromatography, affinity-purification or precipitation. The term “purified” does not require absolute purity; rather, it is intended as a relative term. Thus, for example, a purified polypeptide (or capsomere, or VLP) preparation is one in which the polypeptideis more enriched than it is in its generative environment, for instance within a cell or population of cells in which it is replicated naturally or in an artificial environment. A preparation of substantially pure chimeric L1/L2 polypeptides can be purified such that the desired chimeric polypeptides represent at least 50% of the total protein content of the preparation. In certain embodiments, a chimeric L1/L2 polypeptide will represent at least 60%, at least 70%, at least 80%, at least 85%, at least 90%, or at least 95% or more of the total protein content of the preparation.

In the production and purification of a chimeric L1/L2 polypeptide, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art.

Expressed chimeric L1/L2 polypeptides can then be recovered and purified from recombinant cell cultures by any of a number of methods well known in the art, including ammonium sulfate or ethanol precipitation, acid extraction, filtration, ultrafiltration, centrifugation, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted above, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, U.K.; Scopes (1993) Protein Purification: Principles and Practice, 3^{th} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ. WO2010/012780 (incorporated herein by reference) describes a process for purifying HPV 16 and HPV 18 VLPs. An analgous process can be applied to the purification of the chimeric polypeptides described herein. Thus the chimeric polypeptides may be extracted from host cells in a reducing β-mercaptoethanol (BME) butter and then subjected to anion and hydroxyapatite chromatography and then allowing the resulting product to mature, by BME removal. The resulting product may be rendered sterile, by sterile filtration.

### Immunogenic Compositions and Methods

Another aspect of the present disclosure concerns immunogenic compositions that contain chimeric L1/L2 polypeptides (or capsomeres or VLPs made up of the chimeric L1/L2 polypeptides). Such immunogenic compositions can include the chimeric L1/L2 polypeptides alone or in combination, e.g., with additional chimeric L1/L2 polypeptides and/or with VLPs (for example, L1 VLPs).

In certain embodiments, any of the chimeric L1/L2 polypeptides described hereinabove is a component of the immunogenic composition. For example, the immunogenic composition can include a chimeric L1/L2 polypeptide that includes an HPV type 18 L1 polypeptide or fragment thereof into which at least one peptide comprising an epitope of an L2 polypeptide has been inserted (e.g., a non-HPV type 18 L2 peptide). Similarly, the immunogenic composition can include a chimeric L1/L2 polypeptide that includes an HPV type 16 L1 polypeptide or fragment thereof into which at least one peptide comprising an eptiope of an L2 polypeptide has been inserted (e.g., a non-HPV type 16 L2 peptide). In specific examples, the L2 peptide inserted into the HPV 16 L1 polypeptide includes (e.g., consists of) amino acids 56-75 (as designated with respect to an alignment with HPV 16 L2) of the L2 polypeptide. Additional suitable chimeric L1/L2 polypeptides for use in immunogenic compositions include any of those described above.

In certain embodiments, the chimeric L1/L2 polypeptides are present in immunogenic compositions in combination with HPV VLPs. For example, in one embodiment, the immunogenic composition includes:
(i) at least one virus like particle (VLP) comprising a human papillomavirus (HPV) L1 polypeptide or fragment thereof; and
(ii) at least one chimeric polypeptide comprising a human papillomavirus (HPV) L1 or fragment thereof comprising at least one peptide comprising an epitope of an L2 polypeptide inserted within the HPV L1 polypeptide.

In an embodiment the chimeric polypeptide is a polypeptide as herein described.

In a favoured embodiment at least one VLP comprises a HPV 16 VLP L1 polypeptide or fragment thereof. In another embodiment at least one VLP comprises HPV 18 L1 polypeptide of fragment thereof. Favourably in such compositions the chimeric polypeptide is assembled into supra molecular assembly such as capsomeres or Virus like particles or small non-VLP like structure.

In one embodiment the composition comprises (i) at least one HPV L1 VLP; and (ii) two chimeric HPV L1 VLPs, polypeptides or capsomeres each comprising an L2 peptide in the L1 sequence. In a favoured embodiment the two chimeric L1 polypeptides (or capsomers or VLPs) can comprise different L2 peptides in L1 polypeptides from the same HPV type (for example HPV 16 or HPV 18). Alternatively the two chimeric L1 polypeptides, capsomeres or VLPs can comprise the same L2 peptide in L1 polypeptide from two different HPV types such as HPV 16 and HPV 18. In yet a further embodiment, the two chimeric L1 polypeptides or capsomers or VLPs, can comprise different L2 peptides in L1 polypeptides from two different HPV types (such as HPV 16 and HPV 18 and/or HPV 33 and HPV 58). In specific embodiments, the L2 peptides can be from HPV 33 or HPV 58, and can be inserted into HPV 18 μl. In such embodiments, the different L2 peptides can be inserted singly into two different HPV type 18 L1 polypetpides, or they can be inserted into the same or different sites in the same HPV type 18 L1 polypeptide.

In certain embodiments, the HPV VLPs (particularly HPV L1 only VLPs) and chimeric VLPs, polypeptides or capsomers, included in the compositions according to the present disclosure can include one or more of HPV 6 VLPs, HPV 11 VLPs, HPV 16 VLPs and HPV 18 L1 VLPs. For example they can include VLPs of HPV 16 and 18, or HPV 6 and 11, or of all 4 HPV types. Suitably the HPV L1 VLPs and chimeric HPV L1 polypeptides, are from HPV 16 and/or HPV 18.

HPV VLPs for use as described herein, either chimeric or non-chimeric, can be assembled from L2 also, or they can be L1 only VLPs. For example, VLPs can be assembled from a mixture of L1 and L2 polypeptides (and as such are not the same as the chimeric L1/L2 VLPs disclosed herein, in which an L2 peptide is inserted into the L1 sequence). Alternatively, the VLPs can be chimeric VLPs other than the L1/L2 polypeptide disclosed herein. For example, such non-L1/L2 polypeptides can include an L1 polypeptide and at least one additional sequence of an HPV polypeptide other than L1, such as E7.

The HPV L1 in the VLPs or from the chimeric polypeptides disclosed herein can be formed from either full length HPV L1 protein or certain L1 derivatives, such as fragments, using standard methods in the art, for example as disclosed in WO 03/077942 (U.S. Pat. No. 7,416,846) or WO99/13056 (U.S. Pat. No. 7,351,533) incorporated herein by reference.

In a particular embodiment of the composition disclosed herein, the HPV L1 VLPs comprise or consist of HPV 16 and HPV 18 VLPs, and the chimeric HPV L1 VLPs comprise or consist of chimeric HPV 16 L1 VLPs or chimeric HPV 18 μl VLPs or both. Where both HPV 16 and HPV 18 chimeric L1 VLPs are present, the L2 peptides in each can be the same or different and can be any of the L2 peptides disclosed herein.

The immunogenic compositions disclosed herein typically include at least one pharmaceutically acceptable diluent or carrier and optionally an adjuvant. An immunogenic composition is a composition which raises an immune response when administered to an animal or human, which immune response can be a protective immune response which is not necessarily fully protective against infection or disease but at least reduces incidence of infection or disease.

An adjuvant for use as described herein can comprise an aluminium salt. Also suitable are adjuvants which stimulate a Th1 type response such as 3 de-O-acylated monophosphoryl lipid A (3D MPL) or QS21. Suitably the adjuvant is an aluminium salt, suitably in combination with 3D MPL, such as aluminium hydroxide and 3D MPL. Compositions according to the present disclosure comprising such an adjuvant can be prepared as described for example in WO 00/23105 incorporated herein by reference.

HPV L1 VLPs and chimeric HPV L1 VLPs for use as described herein can be adsorbed on to aluminium containing adjuvants. The adjuvant can be added to the different VLPs to pre-adsorb them before mixing of the different VLPs to form the final vaccine product.

The immunogenic composition can also comprise aluminium or an aluminium compound as a stabiliser, and the present disclosure also relates to a stabilised composition wherein the VLPs are adsorbed onto an aluminium salt. Suitably the VLPs are more stable over time after adsorption onto an aluminium salt than in the absence of aluminium.

The immunogenic compositions described herein can be administered as vaccines by any of a variety of routes such as oral, topical, subcutaneous, musosal (typically intravaginal), intravenous, intramuscular, intranasal, sublingual, intradermal and via suppository. Intramuscular and intradermal deliveries are preferred.

The dosage of the polypeptides, and/or capsomeres and/or VLPs and other proteins can vary with the condition, sex, age and weight of the individual, the administration route and HPV of the vaccine.

The dosage of the polypeptide and/or VLPs present in compositions described herein can vary with the condition, sex, age and weight of the individual, the administration route and HPV of the vaccine. The quantity can also be varied with the number of VLP types. Suitably the delivery is of an amount of VLP suitable to generate an immunologically protective response. Suitably each vaccine dose comprises 1-100 μg of each VLP, suitably at least 5 μg, or at least 10 μg, for example, between 5-50 μg each VLP, most suitably 10-50 μg of each VLP, such as with 5 μg, 6 μg, 10 μg, 15 μg, 20 μg, 40 μg or 50 μg. In certain embodiments, where both chimeric and non-chimeric VLPs are present these amounts reflect the total of the VLPs present for each HPV type i.e. chimeric L1 VLPs with an L2 peptides and L1 VLPs without L2 peptide.

For example a composition according to the present disclosure can comprise, in a single dose:

30 μg HPV 16 VLPs

30 μg HPV 18 VLPs

10 μg chimeric HPV 16 VLPs with L2 peptide

10 μg chimeric HPV 18 VLPs with L2 peptide,

where the L2 peptide in the chimeric HPV 16 and HPV 18 VLPs is the same or different, and can be selected from L2 56-75 and L2 17-36 as described hereinabove, from the same or different HPV types.

In another example a composition according to the present disclosure can comprise, in a single dose:

20 μg HPV 16 VLPs

20 μg HPV 18 VLPs

10-20 μg chimeric HPV 18 VLPs with L2 peptide

10-20 μg chimeric HPV 16 VLPs with L2 peptide,

where the L2 peptide in the chimeric HPV 16 and HPV 18 VLPs is the same or different, and can be selected from L2 56-75 and L2 17-36 as described hereinabove, from the same or different HPV types.

Suitably the compositions above further comprise an adjuvant, suitably an aluminium salt, suitably aluminium hydroxide, suitably in combination with a Th1 adjuvant such as 3D-MPL.

The compositions described herein suitably generate an immune response in a human or animal subject against 1, 2 or more HPV genotypes, suitably any 1, 2 or 3, 4, 5 or more selected from the group of HPV 5, 6, 8, 11, 16, 18, 31, 33, 35, 38, 39, 45, 51, 52, 56, 58, 59, 66, 68 and 73. Favourably the compositions may generate an immune response against one or more of HPV types 2, 3 and 73.

The compositions described herein suitably provide protection against infection and/or disease from 1, 2 or more HPV genotypes, suitably any 1, 2 or more selected from HPV 5, 6, 8, 11, 16, 18, 31, 33, 35, 38, 39, 45, 51, 52, 56, 58, 59, 66, 68 and 73. Suitably the compositions provide protection against at least HPV 16 or 18, and more suitably against both HPV 16 and 18.

Suitably the compositions described herein provide protection against HPV 16 and 18 and at least one other HPV type selected from cancer causing HPV type, genital warts causing HPV types and skin cancer causing HPV types. Suitably the compostions provide protection against one or more of the following HPV types in addition to HPV 16 and HPV 18: HPV 5, 6, 8, 11, 31, 33, 35, 38, 39, 45, 51, 52, 56, 58, 59, 66, 68 and 73.

The immunogenic compositions and vaccines described herein can be used to treat or prevent HPV infection and/or disease. For example the immunogenic composition can be used therapeutically to reduce viral load and/or infections that lead to cervical carcinoma or CIN III sequelae. The present disclosure thus relates to use of the immunogenic compositions described herein in the therapeutic treatment of diseases related to HPV infection and in prophylaxis of infection or disease. Suitably the use of the vaccine of the present disclosure is in prophylaxis of infection and/or disease. The term ‘infection’, as used herein suitably relates to incident infection and/or persistent infection. Infection can be assessed by PCR, for example. The term ‘disease’ as used herein can be abnormal cytology, ASCUS, CIN1, CIN2, CIN3 or cervical cancer related to HPV infection. Disease can be assessed by, for example, histological examination or analysis of biomarkers such as p16.

Optionally the immunogenic composition or vaccine can also be formulated or co-administered with other HPV antigens such as early antigens or non-HPV antigens. Suitably these non HPV antigens can provide protection against other diseases, most suitably sexually transmitted diseases such as herpes simplex virus, chlamydia and HIV. In a particularly embodiment the vaccine comprises gD or a truncate thereof from HSV. In this way the vaccine provides protection against both HPV and HSV.

For all vaccines described herein, the vaccine is suitably used for the vaccination of adolescent girls aged 10-15, suitably 10-13 years. Suitably the vaccine is also suitable for administration to a paediatric population, 0-10 years old. The vaccine can also be administered to women following an abnormal pap smear or after surgery following removal of a lesion caused by HPV. Thus the vaccine is suitably applicable to both a seronegative population as a prophylactic vaccine and/or a seropositive population in a therapeutic setting. The vaccine may also be administered to males.

Suitably the vaccine is delivered in a 2 or 3 dose regimen, for example in a 0, 1 or a 0, 2 or a 0, 3 or a 0, 4 or a 0, 5 or a 0.6 month regimen, or 0, 1 and 6 or a 0, 2, 6 month regimen respectively. Suitably the vaccination regime incorporates a booster injection after 5 to 10 years, suitably 10 years. Other regimes, with 4 or more doses, can also be used.

Suitably the vaccine is a liquid vaccine formulation, although the vaccine can be lyophilised and reconstituted prior to administration.

### EXAMPLES

### Example 1

### Exemplary Chimeric L1/L2 Polypeptides

Expression vectors comprising nucleic acids that encode the following exemplary chimeric L1/L2 polypeptides were produced using molecular biology procedures and summarized in the Table 3.

TABLE 3

| | L1 | Amino acid L1 posi- | L2 peptide | Shown in |
|---|---|---|---|---|
| Chimera # | backbone | tion of L2 peptide | inserted | (SEQ ID NO) |
| #1-L1-HPV18/L2^{DE56-75} | HPV18 | 137-138 | 56-75 HPV58 | (36) |
| #2-L1-HPV18/L2^{Ct56-75} (in NaCl 100 mM) | HPV18 | 432-433 | 56-75 HPV58 | (37) |
| #2-L1-HPV18/L2^{Ct56-75} (in NaCl 500 mM) | HPV18 | 432-433 | 56-75 HPV58 | (37) |
| #3-L1-HPV18/L2^{DE17-36-Ct56-75} (in NaCl 100 mM) | HPV18 | 137-138 | 17-36 HPV33& | (38) |
| | | 432-433 | 56-75 HPV58 | |
| #3-L1-HPV18/L2^{DE17-36-Ct56-75} (in NaCl 500 mM) | HPV18 | 137-138 | 17-36 HPV33& | (38) |
| | | 432-433 | 56-75 HPV58 | |
| #4-L1-HPV16/L2^{Ct56-75} | HPV16 | 431-432 | 56-75 HPV58 | (39) |
| #5-L1-HPV16/L2^{DE17-36} | HPV16 | 137-138 | 17-36 HPV33 | 40 |
| #7-L1-HPV16/L2^{DE17-36-Ct56-75} | HPV16 | 137-138 | 17-36 HPV33& | (42) |
| | | 431-432 | 56-75 HPV58 | |
| #8-L1-HPV18/L2^{DE17-36} | HPV18 | 137-138 | 17-36 HPV33 | (43) |
| #9-L1-HPV18/L2^{Ct17-36} | HPV18 | 432-433 | 17-36 HPV33 | (44) |
| #10-L1-HPV16/L2^{DE56-75} | HPV16 | 137-138 | 56-75 HPV58 | (45) |
| L1-HPV16 | HPV16 | | | FIG. 1a |
| L1-HPV18 | HPV18 | | | FIG. 1b |

Chimera 1: HPV 18 L1 HPV 58 L2 DE chimeric polypeptide wherein the L2 peptide GGLGIGTGSGTGGRTGYVPL (HPV 58/HPV 6) is inserted between position 137 and 138 in a C terminal truncated L1 from HPV 18.
Chimera 2: HPV 18 L1 HPV 58 L2 CT chimeric polypeptide wherein the L2 peptide GGLGIGTGSGTGGRTGYVPL (HPV 58/HPV 6) is inserted between position 432-433 in a C terminal truncated L1 from HPV 18.
Chimera 3: HPV 18 L1 HPV 33 L2 and HPV 58 L2 chimeric polypeptide wherein the L2 peptide QLYQTCKATGTCPPDVIPKV (HPV 33/HPV 11) is inserted between position 137 and 138 and the L2 peptide GGLGIGTGSGTGGRTGYVPL (from HPV 58 or HPV 6) is inserted at position 432-433 in a C terminal truncated L1 from HPV 18.
Chimera 4: HPV 16 L1 HPV 58 L2 CT chimeric polypeptide wherein the L2 peptide GGLGIGTGSGTGGRTGYVPL (HPV 58/HPV 6) is inserted between position 431 & 432 in a C terminal truncated L1 from HPV 16.
Chimera 5: HPV 16 L1 HPV 33 L2 CT chimeric polypeptide wherein the L2 peptide QLYQTCKATGTCPPDVIPKV (HPV 33/HPV 11)is inserted between positions 137 and 138 in a C terminal truncated L1 from HPV 16.
Chimera 6: HPV 16 L1 HPV 33 L2 P/D chimeric polypeptide wherein the L2 peptide QLYQTCKATGTCPPDVIPKV (HPV 33/HPV 11)is inserted between position 272 & 273 in a C terminal truncated L1 from HPV 16.
Chimera 7: HPV 16 L1 HPV 33 L2and HPV 58 L2 chimeric polypeptide wherein the L2 peptide QLYQTCKATGTCPPDVIPKV (HPV 33/HPV 11) is inserted between position 137 and 138 and the L2 peptide GGLGIGTGSGTGGRTGYVPL is inserted at position 431-432 in a C terminal truncated L1 from HPV 16.
Chimera 8: HPV 18 L1 HPV 33 L2 DE chimeric polypeptide wherein the L2 peptide QLYQTCKATGTCPPDVIPKV (HPV 33/HPV 11) is inserted between position 137 and 138 in a C terminal truncated L1 from HPV 18.
Chimera 9: HPV 18 L1 HPV 33 CT chimeric polypeptide wherein the L2 peptide QLYQTCKATGTCPPDVIPKV (HPV 337 HPV 11) is inserted between position 432-433 in a C terminal truncated L1 from HPV 18.
Chimera 10: HPV16 L1 HPV58 L2 chimeric polypeptide wherein the the L2 peptide GGLGIGTGSGTGGRTGYVPL (HPV 58/HPV 6) is inserted between positions 431 and 431 of a C terminal truncated L1 from HPV 16.

### Example 2

### Synthesis Expression, Purification and Characterisation of Exemplary Chimeras

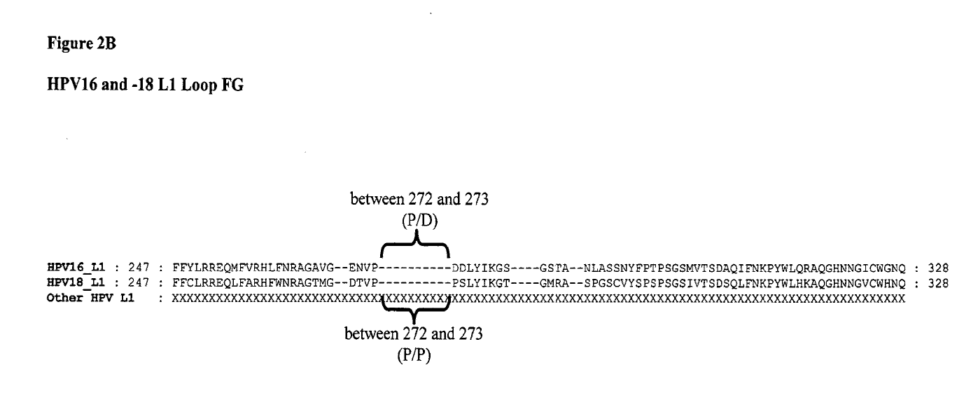

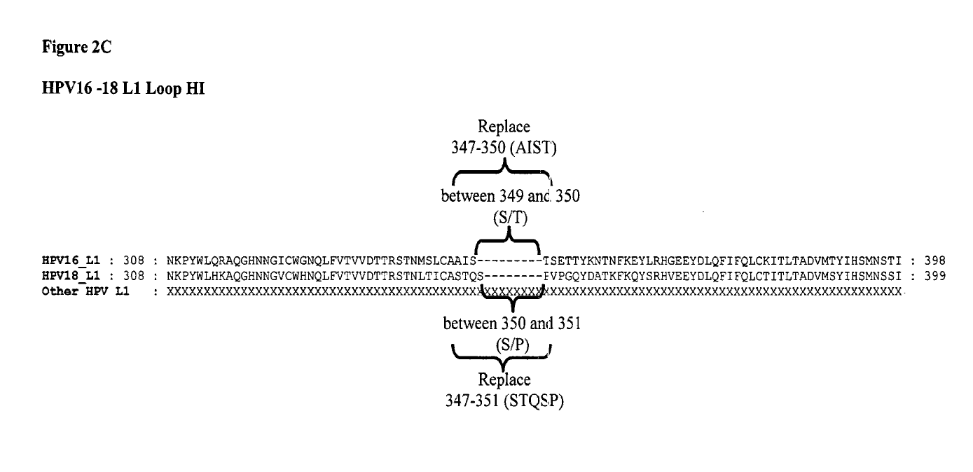

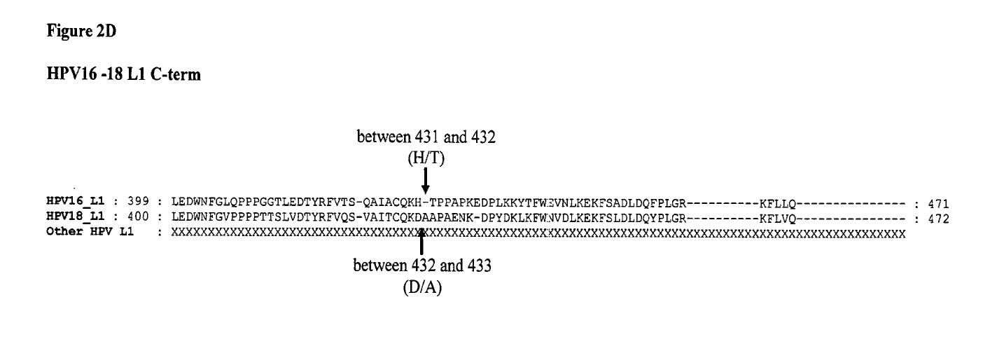

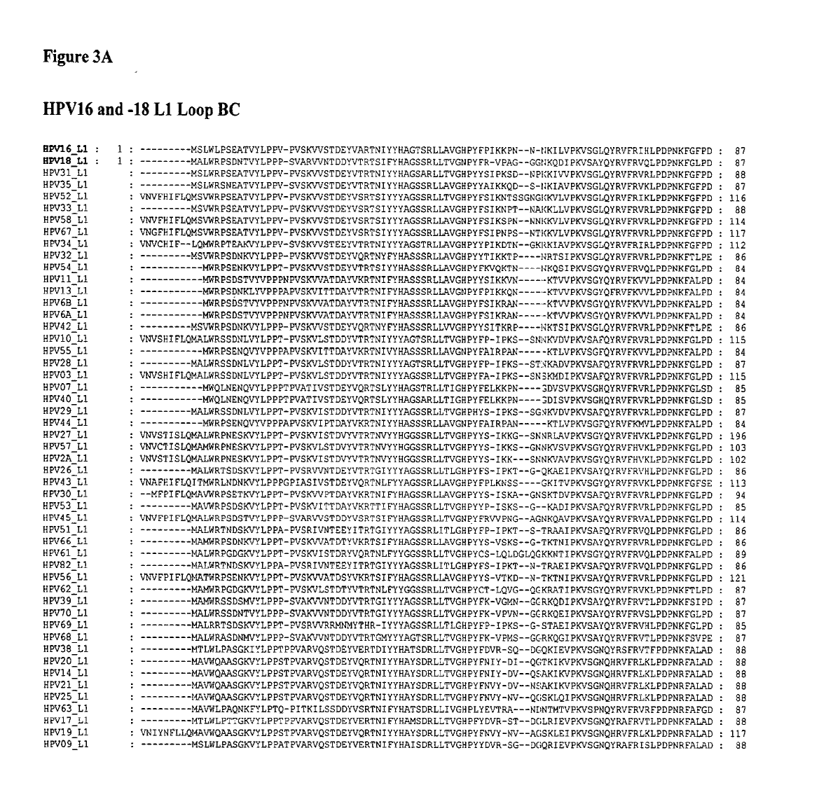

Production of Recombinant Nucleic Acids. Nucleic acids encoding the exemplary chimeric L1/L2 polypeptides described in Example 1 were obtained by gene synthesis prior to their cloning by standard genetic manipulations into a Baculovirus expression vector. The insertion sites are summarized in Table 3. C-terminal truncation of each of the L1 polypeptides aimed at removing the nuclear localization signal, as well as the DNA-binding domain present at the C-terminus of each of the L1 polypeptides (C-terminal end deletions of 34, 35 amino acids, respectively for HPV 16, 18). Amino acid sequences of the HPV 16 and 18 L1 truncates as used herein are shown in FIGS. 1a and b, respectively Amino acid sequences of the HPV 33 and of HVP58 L2 peptides are shown in FIG. 2 (FIG. 2(a), FIG. 2(b), respectively) Amino acid sequences of the exemplary chimeras are provided in SEQ ID NOs: 36-45.

Cell Harvest. The exemplary chimeric polypeptides were expressed in Trichoplusia ni (High Five™) cells (at a density of ˜2 000 000 cells/ml) infected with recombinant Baculovirus (MOI of 0.05-0.5) encoding the HPV 16 or 18 L1/L2 chimeric polypeptides of interest. Cells were harvested at day 4 post infection by low speed centrifugation. The resulting cell pellets were stored at −70° C.

Antigen Extraction. The exemplary chimeric polypeptides were extracted from High

Five™ cells in a two step process of extraction and clarification. The extraction step of cells was performed with a reducing and hypotonic buffer (Tris 20 mM+4% β-mercaptoethanol (BME), pH 8.5). Alternatively, when extraction is low, pH can be 8.7 and detergent Empigen 2% is added. A volume equal to one half or equivalent of culture volume was used to perform the extraction. A contact time of minimum half an hour at room temperature was used. The clarification was performed by centrifugation; if supernatant is turbid, an optional filtration is performed; through a Millistak COHC filter (Millipore) or equivalent.

Purification and Characterization. Purification regimes are very similar for the different chimeric L1/L2 polypeptides, chimeric polypeptides and involve the steps of: Anion exchange chromatography ((Di or Trimethyl amino ethyl—DMAE or TMAE), and Hydroxyapatite chromatography.

Supramolecular formation regimes vary slightly between chimeric polypeptides, differing slightly by NaCl and Tween addition involving the steps of: buffer exchange and BME removal trough gel filtration on Sephadex G25, overnight maturation and 0.22 μm sterilizing filtration.
The purification processes were carried out at room temperature, except for VLP maturation taking place overnight at +4° C. BME 4% v/v was added to all but final buffers in order to prevent VLP formation. All buffers used were filtered on 0.22 μm filters. Prior to each purification run, gel matrixes are sanitised and equilibrated with appropriate buffer before sample loading.
Anion exchange chromatography TMAE or DMAE: The clarified extract was applied to anion exchange column (Di Methyl Amino Ethyl) previouisly equilibrated in Tris 20 mM|NaCl 50 mM|4% β-mercaptoethanol BME buffer, pH 8.0±0.2. After washing for unbound polypeptides, Elution was performed with a linear gradient of Tris 20 mM|NaCl 50-to-250 mM|4% β-mercaptoethanol BME buffer, pH 8.0±0.2. The antigen was eluted within the NaCl gradient and the elution profile was monitored at 280 nm. Fractions collected were analyzed by SDS-PAGE. L1/L2 positive fractions were pooled and kept at +4° C. before next column
Hydroxyapatite chromatography: The eluate of the previous step was applied to a hydroxyapatite Type I (HA) column previously equilibrated in (TRIS 20 mM NaCl-180 mM 4% BME) buffer, pH 8.0±0.2.
After sample application, the gel was washed with equilibration buffer and eluted with approximately 10 column volumes of (Na Phosphate 100 mM|NaCl 30 mM 4% BME) buffer, pH 6.0±0.2. The HA eluate was immediately diluted at up to 40 ml with elution buffer and stored overnight at room temperature.

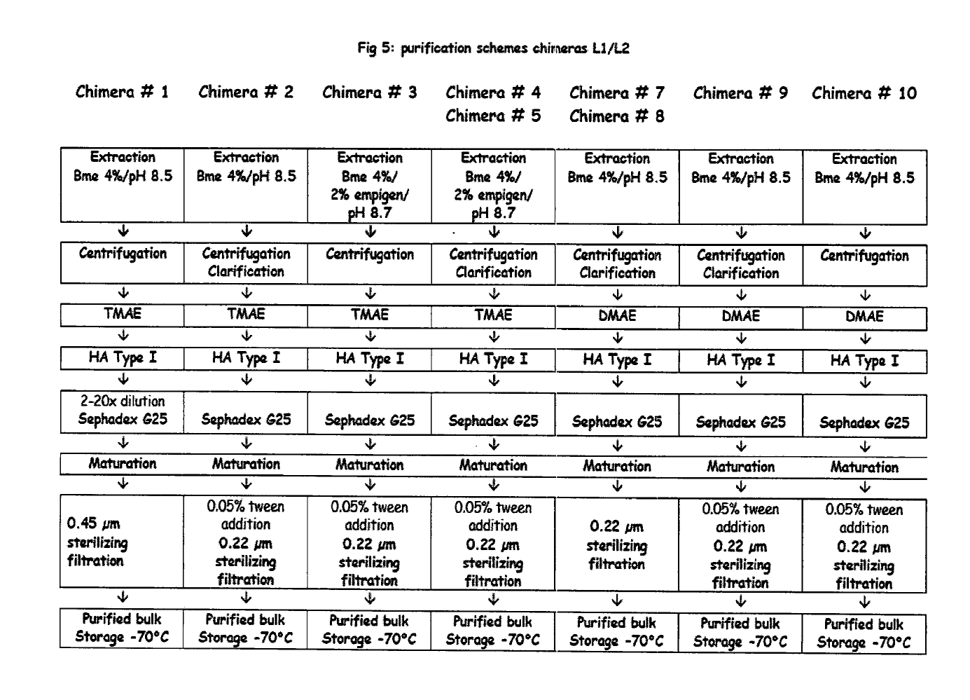

The HA eluates were then applied to a Sephadex G25 (M) gel filtration column (145 ml bed volume) equilibrated in (20 mM Na Phosphate|500 mM of NaCl, pH 6.0) buffer. In certain instances, the buffer was modified to a NaCl content of 100 mM). The elution profiles were monitored at 280 nm (for polypeptide) and 254 nm (for BME). The chimeric L1/L2 antigens are collected in the void volume whereas BME elutes at later stage and with different spectrum from total volume (Vt). Maturation is carried out by overnight storage at +4° C. Following maturation, the Sephadex pools containing the chimeric L1/L2 antigens were filtered, through a 0.22 μm sterile filter and stored at −70° C. In certain cases, 0.05% (V/V) Tween 80 is added prior filtration. A simplified flow chart of a method for purifying chimeric L1/L2 antigens from 800 ml of culture is illustrated in FIG. 5 a)-e).

Electron Microscopy (EM) characterisation of chimeric L1/L2 antigens. Electron microscopy was used to characterise that particles are being formed from the purified chimeric L1/L2 polypeptides, such as polypeptide particles and/or capsomeres and/or VLPs similar to those produced by the C-terminal truncated HPV-16 and HPV-18 L1 proteins used as controls. The size of the chimeric VLP can be smaller or larger than the controls. Purified chimeric L1/L2 polypeptides particles were diluted to 50 μg/ml in their respective buffer (as shown in Table 3). The samples were prepared for EM negative staining analysis according to a standard two-step negative staining method (Hayat M. A. & Miller S. E., 1990) using Uranyl Acetate (UAc) as contrasting agent. Briefly, a nickel grid (400 mesh) with carbon-coated formvar film was floated on a drop of the sample for 10 min at room temperature to allow adsorption of the material. Excess solution was removed and the material let to airdry for less than 2 min. The grid was then briefly (less than 30 sec) floated on a drop of distilled water to remove salts that could yield stain precipitate. The grid was transferred on a drop of stain prepared according to Harris (Harris, J. R., 1994): 2% UAc (w/v) in water, supplemented with 1% trehalose (w/v). The grid was blotted dry after 30 s. The material was left to dry completely (over 1 hr) and examined under the LEO Zeiss EM912Ω at 100 kV. Representative fields were imaged at standard 100K original magnifications and summarized in Table 4 and EM results showed that the chimeric L1/L2 polypeptides formed are not identical to those produced by the wild type HPV-16 or HPV-18 L1 VLP except for #5-L1-HPV16/L2^{DE17-36} and #8-L1-HPV18/L2^{DE17-36}. The particles formed are either under VLP stage, amorphous structures or small and relatively homogenous non-VLP structures.

TABLE 4
Summary of EM results: Chimeric L1/L2 polypeptides

| Chimera | Buffer* | Observations |
|---|---|---|
| #1 | PO4 20 mM- NaCl 500 mM- | Huge amorphous structures |
| | pH 6.0 | |
| #1 | PO4 20 mM- NaCl 500 mM- | Huge amorphous structures |
| | tween 0.05%- pH 6.0 | |
| #2 | PO4 20 mM- NaCl 100 mM- | Amorphous aggregates |
| | tween 0.05% - pH 6.0 | |
| #2 | PO4 20 mM- NaCl 500 mM- | Small non-VLP structures |
| | tween 0.05%- pH 6.0 | |
| #3 | PO4 20 mM- NaCl 100 mM- | Amorphous aggregates |
| | tween 0.05% - pH 6.0 | |
| #3 | PO4 20 mM- NaCl 500 mM- | Small non-VLP structures |
| | tween 0.05%- pH 6.0 | |
| #4 | PO4 20 mM- NaCl 500 mM- | Small non-VLP structures |
| | tween 0.05%- pH 6.0 | |
| #5 | PO4 20 mM- NaCl 500 mM- | VLP |
| | tween 0.05%- pH 6.0 | |
| #7 | PO4 20 mM- NaCl 500 mM- | Small non-VLP structures |
| | pH 6.0 | |
| #8 | PO4 20 mM- NaCl 500 mM- | VLP |
| | pH 6.0 | |
| #9 | PO4 20 mM- NaCl 500 mM- | Small non-VLP structures |
| | tween 0.05%- pH 6.0 | |
| #10 | PO4 20 mM- NaCl 500 mM- | Huge amorphous structures |
| | tween 0.05%- pH 6.0 | |

*Elution buffer

Antibody characterisation of chimeric L1/L2 antigens. Antigenic characterisation of the L1 component of the purified L1/L2 chimeric constructs was carried out by a sandwich ELISA using as coating either H16.V5 (neutralizing and conformation specific monoclonal, antibody; aa 266-297 and 339-365 critical for binding HPV-16 L1 VLPs), H18.J4 (neutralizing and conformation specific monoclonal antibody, (epitope location unknown) on HPV-18 L1 VLPs) or H16.U4 (neutralizing and conformation specific monoclonal, antibody, unknown epitope on HPV-16 L1 VLPs) purified from hybridomas provided by Dr. Neil Christensen (Chistensen et al., 1996, 2001). The assay was used to demonstrate the presence of HPV-16 or HPV-18 conformational specific epitopes on the various chimeric L1/L2 antigens compared to native preparations of HPV-16 or HPV-18 VLPs. The L2 component of the purified L1/L2 chimeric constructs was characterized using a direct ELISA by coating plates with the chimeric constructs followed by detection with either rabbit polyclonal directed to L2 peptide amino acid 17-36 HPV33/HPV11 or 56-75 HPV 58/HPV 6. This assay showed that the L2 epitope is well exposed at surface of the chimeric L1/L2 polypeptide except for #9-L1-HPV 18/L2^{Ct17-36}.

TABLE 5
summarises the data.

| | | | | pAb L2 | pAb L2 |
|---|---|---|---|---|---|
| Chimera # | H16.V5 | H16.U4 | H18.J4 | 17-36 | 56-75 |
| #1-L1-HPV18/L2^{DE56-75} | ND | ND | ND | ND | ND |
| #2-L1-HPV18/L2^{Ct56-75} (in NaCl 100 mM) | ND | ND | + | − | + |
| #2-L1-HPV18/L2^{Ct56-75} (in NaCl 500 mM) | ND | ND | + | − | + |
| #3-L1-HPV18/L2^{DE17-36-Ct56-75} (in NaCl 100 mM) | ND | ND | + | + | + |
| #3-L1-HPV18/L2^{DE17-36-Ct56-75} (in NaCl 500 mM) | ND | ND | − | + | + |
| #4-L1-HPV16/L2^{Ct56-75} | + | ND | ND | − | + |
| #5-L1-HPV16/L2^{DE17-36} | + | + | ND | + | − |
| #7-L1-HPV16/L2^{DE17-36-Ct56-75} | +/− | ND | ND | + | + |
| #8-L1-HPV18/L2^{DE17-36} | ND | ND | − | + | − |
| #9-L1-HPV18/L2^{Ct17-36} | ND | ND | + | − | − |
| #10-L1-HPV16/L2^{DE56-75} | − | ND | ND | − | + |
| HPV 16 L1 VLPs | + | + | − | ND | ND |
| HPV 18 L1 VLPs | − | − | + | ND | ND |

ND: not determined.

### Example 3

### Method for Testing Immunogenicity and Cross Reactivity of Chimeric L1/L2 Antigens in an Animal Model

BALB/c mice (typically, at least 15 mice per group) were immunized intramuscularly (for example, in a multidose regimen of three times at day 0, 14 and 42) with 2 or 10 μg of the aforesaid chimera L1/L2 polypeptide alone or administered with Cervarix, followed by two boosts two and six weeks later. After a suitable period (e.g. on day 14) following the last immunization, the specific L1 antibody responses induced by vaccination were monitored by peptide and/or protein-ELISA. The specific and cross reactive L2 antibody responses induced by vaccination can be monitored by peptide and/or protein-ELISA. ELISA titers were calculated from a reference by SoftMaxPro (using a four parameters equation) and expressed in EU/ml.

Alternatively, Two New Zealand White rabbits (NZW, 1.5-2 kg) were immunized by intramuscular administration with 20 or 100 μg of the aforesaid chimera L1/L2 polypeptide alone or administered with Cervarix (for example, in a multidose regimen of four times at day 0, 14, 28 & 42). The chimeras were formulated with Specol (from Cedi Diagnostic), a water in-oil emulsion used as an alternative to Freund's adjuvant for hyperimmunization of rabbits, prepared according to the manufacturer's protocols.

Anti-VLPs serology (Ig response). Quantification of anti-VLP16 or VLP18 antibody is carried out by ELISA using HPV 16 VLPs or HPV 18 VLPs as a coating antigen.

Table 6 & 7 summarise the data in mice and rabbit, respectively.

TABLE 6
Binding of mouse antiserum induced by chimeric L1/L2 polypeptides to
HPV-16, 18 L1 VLPs on day 14 following the last immunization (ELISA).

| | ELISA : Anti-L1 VLP response (EU/ml) | | | | | |
|---|---|---|---|---|---|---|
| | HPV-16 | | | HPV-18 | | |
| | | LL | UL | | LL | UL |
| Formulation | GMT | CI95 | CI95 | GMT | CI95 | CI95 |
| HPV16/18 L1 VLP 2 μg/AS04 | 1661845 | 1142855 | 2416516 | 2068578 | 1207914 | 3542483 |
| #2-L1-HPV18/L2^{Ct56-75} | 434982 | 317060 | 596763 | 3165739 | 2296242 | 4364479 |
| (in NaCl 100 mM) 10 μg/AS04 | | | | | | |
| #5-L1-HPV16/L2^{DE17-36} 10 μg/AS04 | 1243267 | 912131 | 1694616 | 60647 | 41878 | 87828 |
| #8-L1-HPV18/L2^{DE17-36} 10 μg/AS04 | 197839 | 133820 | 292486 | 1586703 | 1326262 | 1898286 |
| HPV16/18 L1 VLP 2 μg + | 658494 | 505689 | 857472 | 1550403 | 1201194 | 2001135 |
| #2-L1-HPV18/L2^{Ct56-75} | | | | | | |
| (in NaCl 100 mM) 2 μg/AS04 | | | | | | |
| HPV16/18 L1 VLP 2 μg + | 1096750 | 982819 | 1223889 | 2003694 | 1583281 | 2535739 |
| #2-L1-HPV18/L2^{Ct56-75} | | | | | | |
| (in NaCl 100 mM)) 10 μg/AS04 | | | | | | |
| HPV16/18 L1 VLP 2 μg + | 1171974 | 805890 | 1704356 | 1036944 | 700439 | 1535113 |
| #5-L1-HPV16/L2^{DE17-36} 2 μg/AS04 | | | | | | |
| HPV16/18 L1 VLP 2 μg + | 1901428 | 1327269 | 2723962 | 911637 | 653794 | 1271166 |
| #5-L1-HPV16/L2^{DE17-36} 10 μg/AS04 | | | | | | |
| HPV16/18 L1 VLP 2 μg + | 982738 | 779502 | 1238963 | 1504784 | 1210425 | 1870728 |
| #8-L1-HPV18/L2^{DE17-36} 2 μg/AS04 | | | | | | |
| HPV16/18 L1 VLP 2 μg + | 813835 | 631288 | 1049168 | 1261779 | 1061768 | 1499467 |
| #8-L1-HPV18/L2^{DE17-36} 10 μg/AS04 | | | | | | |

LL = Lower limit;
UL = Upper limit,
CI95 = Confidence Interval

100% of the mice sera reacted with HPV16 and HPV18 L1 VLP showing that insertion of L2 epitope did not affect the HPV-L1 response.

TABLE 7
Binding of rabbit antiserum induced by chimeric
L1/L2 polypeptides to HPV-16, 18 L1 VLPs on day
14 following the last immunization (ELISA)

| | | ELISA: Anti-L1 VLP | |
|---|---|---|---|
| | Rabbit | response (EU/ml) | |
| Formulation | id# | HPV-16 | HPV-18 |
| HPV16/18 L1 VLP 2 μg/AS04 | TA368 | 57 037 | 21 995 |
| | TA369 | 108 886 | 104 117 |
| #2-L1-HPV18/L2^{Ct56-75} | TA380 | 108 286 | 984 466 |
| (in NaCl 100 mM)100 μg/Specol | TA381 | 47 879 | 785 301 |
| #5-L1-HPV16/L2^{DE17-36} | TA382 | 165 675 | 6 540 |
| 80 μg/Specol | TA383 | 327 429 | 6 771 |
| #8-L1-HPV18/L2^{DE17-36} | TA370 | 5 049 | 62 505 |
| 100 μg/AS04 | TA371 | 2 851 | 55 907 |
| #8-L1-HPV18/L2^{DE17-36} | TA376 | 16 039 | 688 188 |
| 100 μg/Specol | TA377 | 8 673 | 575 679 |
| HPV16/18 L1 VLP 20 μg + #8-L1- | TA372 | 80 313 | 70 839 |
| HPV18/L2^{DE17-36} 20 μg/AS04 | TA373 | 64 274 | 118 238 |
| HPV16/18 L1 VLP 20 μg + #8-L1- | TA374 | 1 136 885 | 850 922 |
| HPV18/L2^{DE17-36} 20 μg/Specol | TA375 | 396 871 | 474 109 |
| HPV16/18 L1 VLP 20 μg + #8-L1- | TA378 | 385 169 | 622 211 |
| HPV18/L2^{DE17-36} 100 μg/Specol | TA379 | 579 951 | 1 037 924 |

100% of the rabbit sera reacted with HPV16 and HPV18 L1 VLP showing that insertion of L2 epitope did not affect HPV-L1 response.

Anti-L2 peptide serology (Ig response). Quantification of anti-L2 antibody was performed by ELISA using the L2 peptide (2 ug/ml) from the homologous (L2 peptides amino acid 17-36 HPV33/HPV11 or L2 peptides amino acid 56-75 HPV58/HPV6) or heterologous HPV types to assess specific and cross-reactive responses. For the cross L2 antibody response, the following synthetic L2 peptides were used: amino acid 17-36 from HPV-5, 6, 16, 31, 35, 52 and 56 or amino acid 56-75 from HPV-58, 45, 33, 52, 5, 11, 56 and 35.

### HPV-L2-Peptide ELISA Measurement

### HPV-L2-Peptide ELISA Measurement

L2 peptides (produced by Eurogentec) were diluted at a final concentration of 2 μg/ml in PBS and were adsorbed overnight at 4° C. onto the wells of 96-wells microtiter plates (Maxisorp Immuno-plate, Nunc, Denmark). The plates were then incubated for 1 hr at 37° C. with PBS+0.1% Tween20+1% BSA (saturation buffer). Sera diluted in saturation buffer were added to the HPV L2 peptide-coated plates and incubated for 1 hr 30 at 37° C. The plates were washed four times with PBS 0.1% Tween20 and biotin-conjugated anti-Ig diluted in saturation buffer was added to each well and incubated for 1 hr for anti-mouse reagent (Dako, UK) or 1 hr 30 for anti-rabbit reagent (Amersham, UK) at 37° C. After a washing step, streptavidin-horseradish peroxydase (Dako, UK), diluted in saturation buffer was added for an additional 30 min at 37° C. Plates were washed as indicated above and incubated for 20 min at room temperature with a solution of 0.04% o-phenylenediamine (Sigma) 0.03% H₂O₂ in 0.1% Tween20, 0.05M citrate buffer pH 4.5. The reaction was stopped with 2N H2SO4 and read at 492/620 nm. ELISA titers were calculated from a reference by SoftMaxPro (using a four parameters equation) and expressed in EU/ml.

### HPV-L1 ELISA Measurement

HPV-16/18 L1 VLPs were diluted at a final concentration of 1 μg/ml in PBS and were adsorbed overnight at 4° C. onto the wells of 96-wells microtiter plates (Maxisorp Immuno-plate, Nunc, Denmark). The plates were then incubated for 1 hr at 37° C. with PBS+0.1% Tween20+1% BSA (saturation buffer). Sera diluted in saturation buffer were added to the HPV L1 peptide-coated plates and incubated for 1 hr 30 at 37° C. The plates were washed four times with PBS 0.1% Tween20 and biotin-conjugated anti-mouse Ig (Dako, UK) or anti-rabbit Ig (Amersham UK) diluted in saturation buffer was added to each well and incubated for 1 hr 30 at 37° C. After a washing step, streptavidin-horseradish peroxydase (Dako, UK), diluted in saturation buffer was added for an additional 30 min at 37° C. Plates were washed as indicated above and incubated for 20 min at room temperature with a solution of 0.04% o-phenylenediamine (Sigma) 0.03% H2O2 in 0.1% Tween20, 0.05M citrate buffer pH 4.5. The reaction was stopped with 2NH2SO4 and read at 492/620 nm. ELISA titers were calculated from a reference by SoftMaxPro (using a four parameters equation) and expressed in EU/ml.
Table 8 summarises the immunogenicity data in mice.

All chimeric L1/L2 polypeptide antigens induced significant dose dependent L2 antibody responses (titers ranging from 1687-18873) in mice. The results demonstrated improved immunogenicity between post-third and post-fourth immunization (data not shown)

TABLE 8
Binding of mice antiserum induced by chimeric L1/L2 polypeptides
to homologous L2 peptide 17-36 HPV 33 or L2-peptide 56-75
peptide HPV 58 on day 14 following the last immunization (ELISA)

| | ELISA: Anti-L2 peptide response (EU/ml) | | | | | |
|---|---|---|---|---|---|---|
| | L2-peptide 17-36 | | | L2-peptide 56-75 peptide | | |
| | HPV 33 | | | HPV 58 | | |
| Formulation | GMT | LL CI95 | UL CI95 | GMT | LL CI95 | UL CI95 |
| #2-L1-HPV18/L2^{Ct56-75} | NT | NT | NT | 13957 | 9972 | 19533 |
| (in NaCl 100 mM) 10 μg/AS04 | | | | | | |
| #5-L1-HPV16/L2^{DE17-36} 10 μg/AS04 | 6116 | 2953 | 12665 | NT | NT | NT |
| #8-L1-HPV18/L2^{DE17-36} 10 μg/AS04 | 18873 | 11898 | 29937 | NT | NT | NT |
| HPV16/18 L1 VLP 2 μg + | NT | NT | NT | 3199 | 1401 | 7308 |
| #2-L1-HPV18/L2^{Ct56-75} | | | | | | |
| (in NaCl 100 mM) 2 μg /AS04 | | | | | | |
| HPV16/18 L1 VLP 2 μg + | NT | NT | NT | 12653 | 6037 | 26520 |
| #2-L1-HPV18/L2^{Ct56-75} | | | | | | |
| (in NaCl 100 mM) 10 μg/AS04 | | | | | | |
| HPV16/18 L1 VLP 2 μg + | 1687 | 866 | 3285 | NT | NT | NT |
| #5-L1-HPV16/L2^{DE17-36} 2 μg/AS04 | | | | | | |
| HPV16/18 L1 VLP 2 μg + | 2405 | 1565 | 3695 | NT | NT | NT |
| #5-L1-HPV16/L2^{DE17-36} 10 μg/AS04 | | | | | | |
| HPV16/18 L1 VLP 2 μg + | 3965 | 1631 | 9639 | NT | NT | NT |
| #8-L1-HPV18/L2^{DE17-36} 2 μg /AS04 | | | | | | |
| HPV16/18 L1 VLP 2 μg + | 11971 | 7242 | 19786 | NT | NT | NT |
| #8-L1-HPV18/L2^{DE17-36} 10 μg/AS04 | | | | | | |

LL = Lower limit;
UL = Upper limit,
CI95 = Confidence Interval

Table 9 & 10 summarise the ELISA data in rabbit for peptide sequence 17-36 and 56-75 respectively.

All chimeric L1/L2 polypeptide antigens induced significant dose dependent specific and cross-reactive L2 antibody responses in rabbits. The L2 response was specific as the antisera from rabbits immunized with chimera L1/L2 polypeptide containing amino acid 17-36 of L2 did not cross-react with synthetic L2 peptides amino acid 56-75 of L2 and vice versa (data not shown).

A good cross-reactivity was observed with most synthetic L2 peptides from the 17-36 amino acids of L2 sequences (Table 9). The reactivity may be dependant on an amino acid in position 30: a Pro (P) replaced by a different amino acid class such as S or E in all synthetic amino acids with low or no cross reactivity (L2-amino acid 17-36 HPV-31 and -56). Similarly a relatively good cross-reactivity was observed with all synthetic L2 peptides the 56-75 amino acid sequence of L2 (Table 10). The reactivity may be dependant on an amino acid in position 70: a Thr (T) replaced by a different amino acids such as Ala (A) were not cross reactive (L2-amino acid 56-75 HPV-11, -52 and -56).

TABLE 9
Binding of rabbit antiserum induced by chimeric L1/L2 polypeptides to
L2 peptide 17-36 on day 14 following the last immunization (ELISA)

| | Rabbit | ELISA: Anti-peptide L2 peptide 17-36 response (EU/ml) | | | | | | | |
|---|---|---|---|---|---|---|---|---|---|
| Formulation | id# | HPV-5 | HPV-6 | HPV-11* | HPV-16 | HPV-31 | HPV-35 | HPV-52 | HPV-56 |
| #5-L1- | TA382 | 64 367 | 73 243 | 90 769 | 53 601 | 5 410 | 39 251 | 139 671 | 1 362 |
| HPV16/L2^{DE17-36} | TA383 | 77 393 | 160 437 | 154 321 | 70 389 | 2 019 | 49 784 | >216 561 | 900 |
| 80 μg Specol | | | | | | | | | |
| #8-L1- | TA370 | 5 627 | 8 792 | 12 594 | 8 971 | 1 593 | 6 063 | 18 825 | 1 |
| HPV18/L2^{DE17-36} | TA371 | 3 065 | 7 100 | 11 295 | 3 135 | 314 | 2 882 | 17 153 | 84 |
| 100 μg AS04 | | | | | | | | | |
| #8-L1- | TA376 | 33 379 | 74 976 | 109 514 | 38 514 | 3 366 | 29 864 | 151 811 | 3 354 |
| HPV18/L2^{DE17-36} | TA377 | >171 000 | >208 165 | >213 208 | >190 137 | 4 840 | >124 546 | >216 561 | 2 726 |
| 100 μg Specol | | | | | | | | | |
| HPV16/18 | TA372 | 1 533 | 1 370 | 1 633 | 1 517 | 490 | 1 192 | 2 639 | 264 |
| L1 VLP 20 μg + | TA373 | 16 603 | 18 684 | 22 998 | 16 788 | 347 | 14 309 | 29 593 | 253 |
| #8-L1- | | | | | | | | | |
| HPV18/L2^{DE17-36} | | | | | | | | | |
| 20 μg AS04 | | | | | | | | | |
| HPV16/18 | TA374 | 1 277 | 4 484 | 10 479 | 2 832 | 2 362 | 1 689 | 12 252 | 412 |
| L1 VLP 20 μg + | TA375 | 10 217 | 15 197 | 18 916 | 11 226 | 651 | 5 863 | 29 216 | 841 |
| #8-L1- | | | | | | | | | |
| HPV18/L2^{DE17-36} | | | | | | | | | |
| 20 μg Specol | | | | | | | | | |
| HPV16/18 | TA378 | 25 542 | 31 141 | 35 927 | 19 519 | 2 329 | 10 434 | 36 378 | 503 |
| L1 VLP 20 μg + | TA379 | 39 846 | 44 095 | 73 486 | 30 542 | 1 790 | 22 358 | 101 784 | 494 |
| #8-L1- | | | | | | | | | |
| HPV18/L2^{DE17-36} | | | | | | | | | |
| 100 μg Specol | | | | | | | | | |

*17-36 HPV11/HPV33 represents homologous L2 peptide = type specific

TABLE 10
Binding of rabbit antiserum induced by chimeric
L1/L2 polypeptides to L2 peptide 56-75 on day
14 following the last immunization (ELISA)

| | Rabbit | Anti-peptide L2 56-75 response (EU/ml) | | | | | | | |
|---|---|---|---|---|---|---|---|---|---|
| Formulation | id# | HPV-58 | HPV-45 | HPV-33 | HPV-52 | HPV-5 | HPV-11 | HPV-56 | HPV-35 |
| #2 L1/L2 | TA380 | 7 235 | 4 647 | 7 679 | 34 | 2 881 | 48 | 38 | 2 091 |
| (in NaCl 100 mM) | TA381 | 1 065 | 1 201 | 484 | 721 | 550 | 722 | 798 | 934 |
| 100 μg Specol | | | | | | | | | |

*56-75 HPV58/HPV6 represents homologous L2 peptide = type specific

Neutralization experiment. Two weeks after the third and/or fourth immunization, sera were diluted with neutralization assay buffer (8 four-fold serial dilutions—initial dilution started at 1/10 for rabbit sera and 1/40 for mice) and mixed with infectious pseudovirus (PsV) from HPV types which are the same as and different to the one(s) used during immunization. The mixture was reacted for an hour at 4° C. and added to the 293 TT cells (30 000 cells per well) which have been plated at least 2 hours before but not more than 4.5 hours. After culturing for 72 hours with 5% CO₂. The supernatant is recovered and secreted alkaline phosphatase (SeAP) activity measured (Neutralization assay essentially as described in Pastrana et al 2004 modified in that the relative light units were optimised to be in the linear range (e.g. between 75-100 RLU). Neutralizing titers are expressed as the reciprocal of the serum dilution leading to 50% reduction of the SeAP activity signal generated by PsV infection in the absence of serum. Neutralizing titers below 40 are considered below the Cut-off.
Table 11 summarises the data in mice.

TABLE 11
Neutralization of HPV-6, 11, 16, 18, 33, 58 pseudovirions
by antiserum induced by chimeric L1/L2 polypeptides
in mice at day 14 following the third immunization

| | Mice | | | | | | |
|---|---|---|---|---|---|---|---|
| | pool | | | | | | |
| | N^{o} | | | | | | |
| Formulation | (n = 5/ | HPV | HPV | | | HPV | HPV |
| groups | pool) | 6* | 11* | HPV 16 | HPV 18 | 33* | 58* |
| HPV-16/18 L1 VLP 2 ug/AS04 | 10.1 | <40 | <40 | 584 166 | >655 360 | <40 | <40 |
| | 10.2 | <40 | <40 | 170 400 | >655 360 | <40 | <40 |
| | 10.3 | <40 | <40 | 449 492 | 161 388 | <40 | <40 |
| #2-L1-HPV18/L2^{Ct56-75} 100 mM) | 6.1 | <40 | <40 | <40 | 326 306 | <40 | <40 |
| (in NaCl 10 μg/AS04 | 6.2 | <40 | <40 | <40 | 234 682 | <40 | <40 |
| | 6.3 | <40 | <40 | <40 | 337 657 | <40 | <40 |
| #5-L1-HPV16/L2^{DE17-36} 10 μg/AS04 | 9.1 | <40 | 566 | 289 420 | <40 | 1 279 | <40 |
| | 9.2 | <40 | 245 | 271 534 | <40 | 604 | <40 |
| | 9.3 | <40 | 130 | 107 562 | <40 | 424 | 61 |
| #8-L1-HPV18/L2^{DE17-36} 10 μg/AS04 | 3.1 | <40 | 341 | <40 | >655 360 | 314 | <40 |
| | 3.2 | <40 | 943 | <40 | 354 052 | 1 543 | <40 |
| HPV16/18 L1 VLP 2 μg + | 4.1 | <40 | <40 | 555 880 | 214 660 | <40 | <40 |
| #2 L1/L2 (in NaCl 100 mM) | 4.2 | <40 | <40 | 150 417 | 138 334 | <40 | <40 |
| 10 μg/AS04 | 4.3 | <40 | <40 | >655 360 | 73 465 | <40 | <40 |
| HPV16/18 L1 VLP 2 μg + | 7.1 | <40 | <40 | 186 990 | 15 578 | <40 | <40 |
| #5-L1-HPV16/L2^{DE17-36} | 7.2 | <40 | 70 | 253 409 | 85 175 | 208 | 369 |
| 10 μg/AS04 | 7.3 | <40 | 81 | >655 360 | 98 651 | 231 | <40 |
| HPV16/18 L1 VLP 2 μg + | 1.1 | <40 | 368 | 174 526 | 254 121 | 348 | 214 |
| #8-L1-HPV18/L2^{DE17-36} | 1.2 | <40 | 1 134 | >655 360 | 192 239 | 1 026 | <40 |
| 10 μg/AS04 | 1.3 | <40 | 607 | >655 360 | 110 914 | 399 | <40 |

*HPV33/11 and HPV58/6 PsV represents type specific for L2 peptides respectively 17-36 HPV33/HPV11 and L2 peptide 56 75 HPV58/HPV6

Chimeric L1/L2 polypeptide antigens #5-L1-HPV16/L2^{DE17-36} and #8-L1-HPV18/L2^{DE17-36} immunized alone induced detectable specific neutralizing antibodies (titers ranging from 262-696) when tested in mice (Table 10) except #2-L1-HPV18/L2^{Ct56-75}. When combined with HPV16/18 L1 VLPs chimeric L1/L2 polypeptide antigens #5-L1-HPV16/L2^{DE17-36} and #8-L1-HPV18/L2^{DE17-36} induced still detectable neutralizing antibodies but to a lower extent (titers ranging from 61-633).

TABLE 11b
Neutralization of HPV-6, 11, 16, 18, 33, 58 pseudovirions
by antiserum induced by chimeric L1/L2 polypeptides
in mice at day 14 following the third immunization

| | Pseudoneutra titers (ED50) | | | | | |
|---|---|---|---|---|---|---|
| | HPV- | HPV- | HPV- | HPV- | HPV- | HPV- |
| Formulation | 6* | 11* | 16 | 18 | 33* | 58* |
| groups | GMT | GMT | GMT | GMT | GMT | GMT |
| HPV16/18 | <40 | <40 | 355012 | 410781 | <40 | <40 |
| L1 VLP | | | | | | |
| 2 μg /AS04 | | | | | | |
| #2-L1-HPV18/ | <40 | <40 | <40 | 295706 | <40 | <40 |
| L2^{Ct56-75} | | | | | | |
| (in NaCl | | | | | | |
| 100 mM) | | | | | | |
| 10 μg/AS04 | | | | | | |
| #5-L1-HPV16/ | <40 | 262 | 203706 | <40 | 689 | 46 |
| L2^{DE17-36} | | | | | | |
| 10 μg/AS04 | | | | | | |
| #8-L1-HPV18/ | <40 | 567 | <40 | 481697 | 696 | <40 |
| L2^{DE17-36} | | | | | | |
| 10 μg/AS04 | | | | | | |
| HPV16/18 L1 | <40 | <40 | 379827 | 129694 | <40 | <40 |
| VLP 2 μg + | | | | | | |
| #2-L1-HPV18/ | | | | | | |
| L2^{Ct56-75} | | | | | | |
| (in NaCl | | | | | | |
| 100 mM) | | | | | | |
| 10 μg/AS04 | | | | | | |
| HPV16/18 L1 | <40 | 61 | 314321 | 50774 | 124 | 84 |
| VLP 2 μg + | | | | | | |
| #5-L1-HPV16/ | | | | | | |
| L2^{DE17-36} | | | | | | |
| 10 μg/AS04 | | | | | | |
| HPV16/18 L1 | <40 | 633 | 421638 | 175640 | 522 | 70 |
| VLP 2 μg + | | | | | | |
| #8-L1-HPV18/ | | | | | | |
| L2^{DE17-36} | | | | | | |
| 10 μg/AS04 | | | | | | |

*HPV33/11 and HPV58/6 PsV represents type specific for L2 peptides respectively 17-36 HPV33/HPV11 and L2 peptide 56-75 HPV58/HPV6

Table 12 & 13 summarize the neutralization data in rabbit at day 14 post III and post IV respectively.

Most chimeric L1/L2 polypeptide antigens tested induced detectable specific and cross-neutralizing antibodies in rabbits (Table 12 & 13) except L1/L2 chimera#2 (in NaCl 100 mM). The presentation of the L2 peptide 56-75 HPV58/HPV11 may not be optimal to induce neutralizing antibodies despite the observation that this chimeric induced ELISA titers. The insertion of L2 peptides in the chimeric L1/L2 polypeptides did not interfere with the induction of high-titer neutralizing antibodies directed against HPV-16 or HPV-18 L1 (Table 11 &12). Chimeric L1/L2 polypeptides #8 formulated in Specol induced approximately 2 times higher neutralizing titers as compared to Alum-MPL formulation (Table 13) Immunization with the L1/L2 chimera alone induced significantly high neutralizing antibodies to HPV16 or HPV-18 which reflects the antibody response to carrier protein HPV-16 or HPV-18 L1 VLP. Moreover, good neutraliztion of high-risk HPV-33,58 was observed for chimeric L1/L2 polypeptide #5 and to a lesser extend of chimeric L1/L2 polypeptide #8. In addition neutralization of low-risk HPV-6 and 11 was detected for both rabbit sera (Table 13).

TABLE 12
Neutralization of HPV-6, 11, 16, 18, 33, 58 pseudovirions
by antiserum induced by chimeric L1/L2 polypeptides in
rabbits at day 14 following the third immunization.

| | Rabbit | Pseudoneutra titers (ED50) | | | | | |
|---|---|---|---|---|---|---|---|
| Formulation | id# | HPV 6* | HPV 11* | HPV 16 | HPV 18 | HPV 33* | HPV 58* |
| HPV16/18 L1 VLP | TA368 | <40 | <40 | 8 995 | 15 357 | 50 | <40 (<10) |
| 2 μg/AS04 | TA369 | <40 | <40 | 76 400 | 180 798 | <40 (<10) | <40 (<10) |
| L1/L2#2 (in NaCl | TA380 | <40 | <40 | <40 | 221 849 | <40 (<10) | <40 (<10) |
| 100 mM) 100 μg/Specol | TA381 | <40 | <40 | <40 | 216 003 | <40 (<10) | <40 (<10) |
| #5-L1-HPV16/ | TA382 | 3 414 | 428 | 111 733 | 49 | 1 879 | 118 |
| L2^{DE17-36} 80 μg/Specol | TA383 | 2 336 | 6 841 | >163 840 | 69 | 1 167 | 231 |
| #8-L1-HPV18/ | TA370 | <40 | <40 | <40 | 47 215 | <40 (17) | <40 (18) |
| L2^{DE17-36} 100 μg/AS04 | TA371 | <40 | <40 | <40 | 14 778 | 51 | <40 (<10) |
| #8-L1-HPV18/ | TA376 | <40 | 141 | 101 | 114 542 | 306 | 124 |
| L2^{DE17-36} 100 μg/Specol | TA377 | <40 | <40 | 61 | 125 200 | 58 | 51 |
| HPV16/18 L1 VLP | TA372 | 282 | 102 | 28 157 | 76 596 | <40 (12) | 91 |
| 20 μg + #8-L1-HPV18/ | TA373 | 59 | <40 | >163 840 | 129 531 | 140 | <40 (23) |
| L2^{DE17-36} 20 μg/AS04 | | | | | | | |
| HPV16/18 L1 VLP | TA374 | 2 319 | 198 | >163 840 | 235 557 | 336 | 141 |
| 20 μg + #8-L1-HPV18/ | TA375 | <40 | <40 | >163 840 | 91 991 | 44 | <40 (<10) |
| L2^{DE17-36} 20 μg/Specol | | | | | | | |
| HPV16/18 L1 VLP | TA378 | <40 | 135 | >163 840 | 197 836 | 292 | <40 (22) |
| 20 μg + #8-L1-HPV18/ | TA379 | 242 | <40 | >163 840 | >163 840 | 416 | 188 |
| L2^{DE17-36} 100 μg/Specol | | | | | | | |

*HPV33/11 and HPV58/6 PsV represents type specific for L2 peptides respectively 17-36 HPV33/HPV11 and L2 peptide 56-75 HPV58/HPV6

TABLE 13
Neutralization of HPV-6, 11, 33, 58 pseudovirions by antiserum induced by chimeric
L1/L2 polypeptides in rabbits at day 14 following the fourth immunization

| | Rabbit | Pseudoneutra titers (ED50) | | | |
|---|---|---|---|---|---|
| Formulation | id | HPV 6* | HPV 11* | HPV 33* | HPV 58* |
| HPV16/18 L1 VLP 2 μg/AS04 | TA368 | <40 | <40 | 47 | <40 |
| | TA369 | <40 | <40 | <40 | <40 |
| #2-L1-HPV18/L2^{Ct56-75} (in NaCl 100 mM) | TA380 | <40 | <40 | <40 | <40 |
| 100 μg/Specol | TA381 | <40 | <40 | <40 | <40 |
| #5-L1-HPV16/L2^{DE17-36} 80 μg/Specol | TA382 | 5 303 | 1 608 | 2 287 | 4 784 |
| | TA383 | 2 814 | 8 617 | 4 640 | 2 448 |
| #8-Ll -HPV18/L2^{DE17-36} 100 μg/AS04 | TA370 | <40 | 64 | 99 | 768 |
| | TA371 | 43 | 359 | 667 | 1 019 |
| #8-L1-HPV18/L2^{DE17-36} 100 μg/Specol | TA376 | <40 | 345 | 641 | 3 436 |
| | TA377 | <40 | 439 | 605 | 2 688 |
| HPV16/18 L1 VLP 20 μg + | TA372 | 288 | 89 | <40 | 74 |
| #8-L1-HPV18/L2^{DE17-36} 20 μg/AS04 | TA373 | <40 | <40 | <40 | <40 |
| HPV16/18 L1 VLP 20μg + | TA374 | 2 623 | 1 113 | 2 455 | 2 400 |
| #8-L1-HPV18/L2^{DE17-36} 20 μg/Specol | TA375 | 282 | 76 | 113 | 180 |
| HPV16/18 L1 VLP 20 μg + | TA378 | 119 | 412 | 1 141 | 1 331 |
| #8-L1-HPV18/L2^{DE17-36} 100 μg/Specol | TA379 | 186 | 475 | 1 163 | 2 201 |

*HPV33/11 and HPV58/6 PsV represents type specific for L2 peptides respectively 17-36 HPV33/HPV11 and L2 peptide 56-75 HPV58/HPV6

In another experiment Chimera #2 purified as a small non-VLP and Chimera 3 were formulated into AS04 adjuvant (alum 3D-MPL) and administered separately to Rabbit and tested in Elisa for antipeptide 17-36, and antipeptide 56-75 and anti HPV L1 response. After the second immunisation the following data was obtained.

TABLE 14
Immunogenicity in Rabbit of L1/L2 Chimera-anti L2¹⁷⁻³⁶ (second immunisation)

| | Anti-Peptide L2¹⁷⁻³⁶ Ab Titers | | | | | |
|---|---|---|---|---|---|---|
| | HPV-6 | HPV-11 | HPV-16 | HPV-35 | HPV-52 | HPV-59 |
| #3-L1-HPV18^{DE 17-36/Ct56-75} 20 μg/AS04 | 1033 | 2137 | 1245 | 718 | 2328 | 114 |
| #3-L1-HPV18^{DE 17-36/Ct56-75} 100 μg/AS04 | 608 | 1323 | 484 | 769 | 883 | 87 |
| #3-L1-HPV18^{DE 17-36/Ct56-75} 20 μg/ | 851 | 2035 | 830 | 923 | 2088 | 69 |
| Prime AS04-boost Alum | | | | | | |
| #8-L1-HPV18^{DE 17-36} 100 μg/AS04 | NT | 2076 | NT | NT | NT | NT |

TABLE 15
Immunogenicity in Rabbit of L1/L2 Chimera - -
anti L2⁵⁶⁻⁷⁵ (second immunisation)

| | Anti-Peptide | | | | |
|---|---|---|---|---|---|
| | L2⁵⁶⁻⁷⁵ Ab Titers | | | | |
| | HPV- | HPV- | HPV- | HPV- | HPV- |
| | 5 | 11 | 33 | 45 | 58 |
| #2-L1-HPV18^{Ct 56-75} 20 μg/AS04 | 248 | 144 | 532 | 553 | 853 |
| #2-L1-HPV18^{Ct 56-75} 100 μg/AS04 | 15 | 465 | 170 | 138 | 135 |
| #3-L1-HPV18^{DE 17-36/Ct 56-75} | 21 | 22 | 55 | 77 | 73 |
| 20 μg/AS04 | | | | | |
| #3-L1-HPV18^{DE 17-36/Ct 56-75} | 79 | 1296 | 809 | 645 | 141 |
| 100 μg/AS04 | | | | | |
| #3-L1-HPV18^{DE 17-36/Ct 56-75} 20 μg/ | 54 | 460 | 232 | 93 | 2061 |
| AS04/Prime AS04 - boost Alum | | | | | |

TABLE 16
Immunogenicity in Rabbit of L1/L2 Chimera -
anti HPV 18 L1 (second immunisation)

| | Anti-HPV-18 L1 Ab Titers |
|---|---|
| #2-L1-HPV18^{Ct 56-75} 20 μg/AS04 | 86573 |
| #2-L1-HPV18^{Ct 56-75} 100 μg/AS04 | 88567 |
| #3-L1-HPV18^{DE 17-36/Ct 56-75} 20 μg/AS04 | 58734 |
| #3-L1-HPV18^{DE 17-36/Ct 56-75} 100 μg/AS04 | 67833 |
| #3-L1-HPV18^{DE 17-36/Ct 56-75} 20 μg/ | 40754 |
| Prime AS04 - boost Alum | |

### APPENDIX: LIST OF REFERENCES

Alphs H H, Gambhira R, Karanam B, Roberts J N, Jagu S, Schiller J T, Zeng W, Jackson D C, Roden R B. Protection against heterologous human papillomavirus challenge by a synthetic lipopeptide vaccine containing a broadly cross-neutralizing epitope of L2. Proc Natl Acad Sci U S A. 2008 Apr. 15; 105(15):5850-5. Epub 2008 Apr. 14.
Benson D A, Karsch-Mizrachi I, Lipman D J, Ostell J, Wheeler D L. GenBank. Nucleic Acids Res. 36(Database issue):D25-30 (2008).
Bishop B, Dasgupta J, Klein M, Garcea R L, Christensen N D, Zhao R, Chen X S. Crystal structures of four types of human papillomavirus L1 capsid proteins: understanding the specificity of neutralizing monoclonal antibodies. J Biol Chem. 2007 Oct. 26; 282(43):31803-11. Epub 2007 Sep. 4.
Boeckmann B., Bairoch A., Apweiler R., Blatter M.-C., Estreicher A., Gasteiger E., Martin M. J., Michoud K., O'Donovan C., Phan I., Pilbout S., Schneider M. The SWISS-PROT protein knowledgebase and its supplement TrEMBL in 2003. Nucleic Acids Res. 31:365-370 (2003)
Carter J J, Wipf G C, Benki S F, Christensen N D, Galloway D A. Identification of a human papillomavirus type 16-specific epitope on the C-terminal arm of the major capsid protein L1. J. Virol. 2003 November; 77(21):11625-32.
Carter J J, Wipf G C, Madeleine M M, Schwartz S M, Koutsky L A, Galloway D A. Identification of human papillomavirus type 16 L1 surface loops required for neutralization by human sera. J Virol. 2006 May; 80(10):4664-72.
Chen X. S, Garcea R. L, Goldberg I, Casini G, Harrison S. C. Structure of small virus-like particles assembled from the L1 protein of human papillomavirus 16. Mol Cell 2000, 5, 557-567
Christensen N D, Cladel N M, Reed C A, Budgeon L R, Embers M E, Skulsky D M, McClements W L, Ludmerer S W, Jansen K U. Hybrid papillomavirus L1 molecules assemble into virus-like particles that reconstitute conformational epitopes and induce neutralizing antibodies to distinct HPV types. Virology. 2001 Dec. 20; 291(2):324-34.
Christensen N D, Dilner J, Eklund C, Carter J J, Wipf G C, Reed C A, Cladel, N M, Galloway D A Surface, Linear and Conformational epitopes on HPV-16 and HPV-18 virus-like particles as defined by monoclonal antibodies, Virology, 223, 174-184 (1996).
Combita A L, Touzé A, Bousarghin L, Christensen N D, Coursaget P. Identification of two cross-neutralizing linear epitopes within the L1 major capsid protein of human papillomaviruses. J Virol. 2002 July; 76(13):6480-6.
Day P M, Thompson C D, Buck C B, Pang Y Y, Lowy D R, Schiller J T. Neutralization of human papillomavirus with monoclonal antibodies reveals different mechanisms of inhibition. J Virol. 2007 August; 81(16):8784-92. Epub 2007 Jun. 6.
Deschuyteneer M, Elouahabi A, Plainchamp D, Plisnier M, Soete D, Corazza Y, Lockman L, Giannini S and Deschamps M. Molecular and structural characterization of the L1-Virus Like Particles that are used as vaccine antigens in Cervarix™, the AS04-adjuvanted HPV-16 and -18 cervical cancer vaccine
Dessy F J, Giannini S L, Bougelet C A, Kemp T J, David M P, Poncelet S M, Pinto L A, Wettendorff M A. Correlation between direct ELISA, single epitope-based inhibition ELISA and pseudovirion-based neutralization assay for measuring anti-HPV-16 and anti-HPV-18 antibody response after vaccination with the AS04-adjuvanted HPV-16/18 cervical cancer vaccine. Hum Vaccin. 2008 November-December; 4(6):425-34. Epub 2008 Nov. 11.
Embers M E, Budgeon L R, Pickel M, Christensen N D. Protective immunity to rabbit oral and cutaneous papillomaviruses by immunization with short peptides of L2, the minor capsid protein. J Virol. 2002 October; 76(19):9798-805.
Gambhira R, Karanam B, Jagu S, Roberts J N, Buck C B, Bossis I, Alphs H, Culp T, Christensen N D, Roden R B. A protective and broadly cross-neutralizing epitope of human papillomavirus L2. J Virol. 2007 December; 81(24):13927-31. Epub 2007 Oct. 10.
Harris, J. R., 1994, Proc. ICEM XIII, Les Editions de Physique, ed., p. 557
Hayat M. A. & Miller S. E., 1990, Negative Staining, Mc Graw-Hill ed
Kawana K, Matumoto K, Yoshikawa H, Kawana T, Yoshiike K, Kanda T. A surface immunodeterminant of human papillomavirus type 16 minor capsid protein L2. Virology 1998, 245, 353-359
Kawana K, Yoshikawa H, Takentani Y, Yoshiike K, Kanda T. In vitro construction of pseudovirions of human papillomavirus type 16: incorporation of plasmid DNA into reassembled L1/L2 capsids. Journal of Virology 1999, 73, 6188-6190.
Kawana K, Kawana Y, Yoshikawa H, Taketani Y, Yoshiike K, Kanda T. Nasal immunization of mice with peptide having a cross-neutralization epitope on minor capsid protein L2 of human papillomavirus type 16 elicit systemic and mucosal antibodies. Vaccine. 2001 Jan. 8; 19(11-12):1496-502.
Kawana K, Yasugi T, Kanda T, Kino N, Oda K, Okada S, Kawana Y, Nei T, Takada T, Toyoshima S, Tsuchiya A, Kondo K, Yoshikawa H, Tsutsumi O, Taketani Y. Safety and immunogenicity of a peptide containing the cross-neutralization epitope of HPV16 L2 administered nasally in healthy volunteers. Vaccine. 2003 Oct. 1; 21(27-30):4256-60.
Kondo K, Ishii Y, Ochi H, Matsumoto T, Yoshikawa H, Kanda T. Neutralization of HPV16, 18, 31, and 58 pseudovirions with antisera induced by immunizing rabbits with synthetic peptides representing segments of the HPV16 minor capsid protein L2 surface region. Virology. 2007 Feb. 20; 358(2):266-72. Epub 2006 Sep. 28.
Kondo K, Ochi H, Matsumoto T, Yoshikawa H, Kanda T. Modification of human papillomavirus-like particle vaccine by insertion of the cross-reactive L2-epitopes. J Med Virol. 2008 May; 80(5):841-6.
Pastrana D V, Buck C B, Pang Y Y, Thompson C D, Castle P E, FitzGerald P C, Krüger Kjaer S, Lowy D R, Schiller J T. Reactivity of human sera in a sensitive, high-throughput pseudovirus-based papillomavirus neutralization assay for HPV16 and HPV18. Virology. 2004 Apr. 10; 321(2):205-16.
Mandavi A, Monk B J. Vaccines against human papillomavirus and cervical cancer: promises and challenges. Oncologist. 2005 August; 10(7):528-38. Review.
Quint W G, Pagliusi S R, Lelie N, de Villiers E M, Wheeler C M; World Health Organization Human Papillomavirus DNA International Collaborative Study Group. Results of the first World Health Organization international collaborative study of detection of human papillomavirus DNA Results of the first World Health Organization international collaborative study of detection of human papillomavirus DNA. J Clin Microbiol. 2006 February; 44(2):571-9
Sadeyen J. R, Tourne S, Shkreli M, Sizaret P. Y, Coursaget P. Insertion of a foreign sequence on capsid surface loops of human papillomavirus type 16 virus-like particles reduces their capacity to induce neutralizing antibodies and delineates a conformational neutralizing epitope. Virology 309 (2003) 32-40
Slupetzky K, Shafti-Keramat, Lenz P, Brandt S, Grassauer A, Sara M and Kirnbauer R. Chimeric papillomavirus-like particles expressing a foreign epitope on capsid surface loops. Journal of General Virology 2001, 82, 2799-2804
Slupetzky K, Gambhira R, Culp T, Shafti-Keramat S, Schellenbacher C, Christensen N. D, Roden R, Kirnbauer R. A papillomavirus-like particle (VLP) vaccine displaying HPV16 L2 epitopes induces cross-neutralizing antibodies to HPV11. Vaccine 25 (2007) 2001-2010
Varsani A, Williamson A L, de Villiers D, Becker I, Christensen N D, Rybicki E P. Chimeric human papillomavirus type 16 (HPV-16) L1 particles presenting the common neutralizing epitope for the L2 minor capsid protein of HPV-6 and HPV-16. J Virol. 2003 August; 77(15):8386-93.
Y.-F. Xul, Y.-Q. Zhang2, X.-M. Xul, and G.-X. Song1. Papillomavirus virus-like particles as vehicles for the delivery of epitopes or genes. Arch Virol (2006) 151: 2133-2148

### Amino Acid Sequences of Exemplary Chimeric L1/L2 Polypeptides

| HPVchim01 | |
|---|---|
| (SEQ ID NO: 36) | |
| 1 | MALWRPSDNT VYLPPPSVAR VVNTDDYVTR TSIFYHAGSS RLLTVGNPYF RVPAGGGNKQ |
| 61 | DIPKVSAYQY RVFRVQLPDP NKFGLPDNSI YNPETQRLVW ACVGVEIGRG QPLGVGLSGH |
| 121 | PFYNKLDDTE SSHAATSGGL GIGTGSGTGG RTGYVPLNVS EDVRDNVSVD YKQTQLCILG |
| 181 | CAPAIGEHWA KGTACKSRPL SQGDCPPLEL KNTVLEDGDM VDTGYGAMDF STLQDTKCEV |
| 241 | PLDICQSICK YPDYLQMSAD PYGDSMFFCL RREQLFARHF WNRAGTMGDT VPPSLYIKGT |
| 301 | GMRASPGSCV YSPSPSGSIV TSDSQLFNKP YWLHKAQGHN NGVCWHNQLF VTVVDTTRST |
| 361 | NLTICASTQS PVPGQYDATK FKQYSRHVEE YDLQFIFQLC TITLTADVMS YIHSMNSSIL |
| 421 | EDWNFGVPPP PTTSLVDTYR FVQSVAITCQ KDAAPAENKD PYDKLKFWNV DLKEKFSLDL |
| 481 | DQYPLGRKFL VQ |
| HPVchim02 | |
| (SEQ ID NO: 37) | |
| 1 | MALWRPSDNT VYLPPPSVAR VVNTDDYVTR TSIFYHAGSS RLLTVGNPYF RVPAGGGNKQ |
| 61 | DIPKVSAYQY RVFRVQLPDP NKFGLPDNSI YNPETQRLVW ACVGVEIGRG QPLGVGLSGH |
| 121 | PFYNKLDDTE SSHAATSNVS EDVRDNVSVD YKQTQLCILG CAPAIGEHWA KGTACKSRPL |
| 181 | SQGDCPPLEL KNTVLEDGDM VDTGYGAMDF STLQDTKCEV PLDICQSICK YPDYLQMSAD |
| 241 | PYGDSMFFCL RREQLFARHF WNRAGTMGDT VPPSLYIKGT GMRASPGSCV YSPSPSGSIV |
| 301 | TSDSQLFNKP YWLHKAQGHN NGVCWHNQLF VTVVDTTRST NLTICASTQS PVPGQYDATK |
| 361 | FKQYSRHVEE YDLQFIFQLC TITLTADVMS YIHSMNSSIL EDWNFGVPPP PTTSLVDTYR |
| 421 | FVQSVAITCQ KDGGLGIGTG SGTGGRTGYV PLAAPAENKD PYDKLKFWNV DLKEKFSLDL |
| 481 | DQYPLGRKFL VQ |
| HPVchim03 | |
| (SEQ ID NO: 38) | |
| 1 | MALWRPSDNT VYLPPPSVAR VVNTDDYVTR TSIFYHAGSS RLLTVGNPYF RVPAGGGNKQ |
| 61 | DIPKVSAYQY RVFRVQLPDP NKFGLPDNSI YNPETQRLVW ACVGVEIGRG QPLGVGLSGH |
| 121 | PFYNKLDDTE SSHAATSQLY QTCKATGTCP PDVIPKVNVS EDVRDNVSVD YKQTQLCILG |
| 181 | CAPAIGEHWA KGTACKSRPL SQGDCPPLEL KNTVLEDGDM VDTGYGAMDF STLQDTKCEV |
| 241 | PLDICQSICK YPDYLQMSAD PYGDSMFFCL RREQLFARHF WNRAGTMGDT VPPSLYIKGT |
| 301 | GMRASPGSCV YSPSPSGSIV TSDSQLFNKP YWLHKAQGHN NGVCWHNQLF VTVVDTTRST |
| 361 | NLTICASTQS PVPGQYDATK FKQYSRHVEE YDLQFIFQLC TITLTADVMS YIHSMNSSIL |
| 421 | EDWNFGVPPP PTTSLVDTYR FVQSVAITCQ KDGGLGIGTG SGTGGRTGYV PLAAPAENKD |
| 481 | PYDKLKFWNV DLKEKFSLDL DQYPLGRKFL VQ |
| HPVchim04 | |
| (SEQ ID NO: 39) | |
| 1 | MSLWLPSEAT VYLPPVPVSK VVSTDEYVAR TNIYYHAGTS RLLAVGHPYF PIKKPNNNKI |
| 61 | LVPKVSGLQY RVFRIHLPDP NKFGFPDTSF YNPDTQRLVW ACVGVEVGRG QPLGVGISGH |
| 121 | PLLNKLDDTE NASAYAANAG VDNRECISMD YKQTQLCLIG CKPPIGEHWG KGSPCTNVAV |
| 181 | NPGDCPPLEL INTVIQDGDM VDTGFGAMDF TTLQANKSEV PLDICTSICK YPDYIKMVSE |
| 241 | PYGDSLFFYL RREQMFVRHL FNRAGAVGEN VPDDLYIKGS GSTANLASSN YFPTPSGSMV |
| 301 | TSDAQIFNKP YWLQRAQGHN NGICWGNQLF VTVVDTTRST NMSLCAAIST SETTYKNTNF |
| 361 | KEYLRHGEEY DLQFIFQLCK ITLTADVMTY IHSMNSTILE DWNFGLQPPP GGTLEDTYRF |
| 421 | VTSQAIACQK HGGLGIGTGS GTGGRTGYVP LTPPAPKEDP LKKYTFWEVN LKEKFSADLD |
| 481 | QFPLGRKFLL Q |
| HPVchim05 | |
| (SEQ ID NO: 40) | |
| 1 | MSLWLPSEAT VYLPPVPVSK VVSTDEYVAR TNIYYHAGTS RLLAVGHPYF PIKKPNNNKI |
| 61 | LVPKVSGLQY RVFRIHLPDP NKFGFPDTSF YNPDTQRLVW ACVGVEVGRG QPLGVGISGH |
| 121 | PLLNKLDDTE NASAYAAQLY QTCKATGTCP PDVIPKVNAG VDNRECISMD YKQTQLCLIG |
| 181 | CKPPIGEHWG KGSPCTNVAV NPGDCPPLEL INTVIQDGDM VDTGFGAMDF TTLQANKSEV |
| 241 | PLDICTSICK YPDYIKMVSE PYGDSLFFYL RREQMFVRHL FNRAGAVGEN VPDDLYIKGS |
| 301 | GSTANLASSN YFPTPSGSMV TSDAQIFNKP YWLQRAQGHN NGICWGNQLF VTVVDTTRST |
| 361 | NMSLCAAIST SETTYKNTNF KEYLRHGEEY DLQFIFQLCK ITLTADVMTY IHSMNSTILE |
| 421 | DWNFGLQPPP GGTLEDTYRF VTSQAIACQK HTPPAPKEDP LKKYTFWEVN LKEKFSADLD |
| 481 | QFPLGRKFLL Q |
| HPVchim06 | |
| (SEQ ID NO: 41) | |
| 1 | MSLWLPSEAT VYLPPVPVSK VVSTDEYVAR TNIYYHAGTS RLLAVGHPYF PIKKPNNNKI |
| 61 | LVPKVSGLQY RVFRIHLPDP NKFGFPDTSF YNPDTQRLVW ACVGVEVGRG QPLGVGISGH |
| 121 | PLLNKLDDTE NASAYAANAG VDNRECISMD YKQTQLCLIG CKPPIGEHWG KGSPCTNVAV |
| 181 | NPGDCPPLEL INTVIQDGDM VDTGFGAMDF TTLQANKSEV PLDICTSICK YPDYIKMVSE |
| 241 | PYGDSLFFYL RREQMFVRHL FNRAGAVGEN VPQLYQTCKA TGTCPPDVIP KVDDLYIKGS |
| 301 | GSTANLASSN YFPTPSGSMV TSDAQIFNKP YWLQRAQGHN NGICWGNQLF VTVVDTTRST |
| 361 | NMSLCAAIST SETTYKNTNF KEYLRHGEEY DLQFIFQLCK ITLTADVMTY IHSMNSTILE |
| 421 | DWNFGLQPPP GGTLEDTYRF VTSQAIACQK HTPPAPKEDP LKKYTFWEVN LKEKFSADLD |
| 481 | QFPLGRKFLL Q |
| HPVchim07 | |
| (SEQ ID NO: 42) | |
| 1 | MSLWLPSEAT VYLPPVPVSK VVSTDEYVAR TNIYYHAGTS RLLAVGHPYF PIKKPNNNKI |
| 61 | LVPKVSGLQY RVFRIHLPDP NKFGFPDTSF YNPDTQRLVW ACVGVEVGRG QPLGVGISGH |
| 121 | PLLNKLDDTE NASAYAAQLY QTCKATGTCP PDVIPKVNAG VDNRECISMD YKQTQLCLIG |
| 181 | CKPPIGEHWG KGSPCTNVAV NPGDCPPLEL INTVIQDGDM VDTGFGAMDF TTLQANKSEV |
| 241 | PLDICTSICK YPDYIKMVSE PYGDSLFFYL RREQMFVRHL FNRAGAVGEN VPDDLYIKGS |
| 301 | GSTANLASSN YFPTPSGSMV TSDAQIFNKP YWLQRAQGHN NGICWGNQLF VTVVDTTRST |
| 361 | NMSLCAAIST SETTYKNTNF KEYLRHGEEY DLQFIFQLCK ITLTADVMTY IHSMNSTILE |
| 421 | DWNFGLQPPP GGTLEDTYRF VTSQAIACQK HGGLGIGTGS GTGGRTGYVP LTPPAPKEDP |
| 481 | LKKYTFWEVN LKEKFSADLD QFPLGRKFLL Q |
| HPVchim08 | |
| (SEQ ID NO: 43) | |
| 1 | MALWRPSDNT VYLPPPSVAR VVNTDDYVTR TSIFYHAGSS RLLTVGNPYF RVPAGGGNKQ |
| 61 | DIPKVSAYQY RVFRVQLPDP NKFGLPDNSI YNPETQRLVW ACVGVEIGRG QPLGVGLSGH |
| 121 | PFYNKLDDTE SSHAATSQLY QTCKATGTCP PDVIPKVNVS EDVRDNVSVD YKQTQLCILG |
| 181 | CAPAIGEHWA KGTACKSRPL SQGDCPPLEL KNTVLEDGDM VDTGYGAMDF STLQDTKCEV |
| 241 | PLDICQSICK YPDYLQMSAD PYGDSMFFCL RREQLFARHF WNRAGTMGDT VPPSLYIKGT |
| 301 | GMRASPGSCV YSPSPSGSIV TSDSQLFNKP YWLHKAQGHN NGVCWHNQLF VTVVDTTRST |
| 361 | NLTICASTQS PVPGQYDATK FKQYSRHVEE YDLQFIFQLC TITLTADVMS YIHSMNSSIL |
| 421 | EDWNFGVPPP PTTSLVDTYR FVQSVAITCQ KDAAPAENKD PYDKLKFWNV DLKEKFSLDL |
| 481 | DQYPLGRKFL VQ |
| HPVchim09 | |
| (SEQ ID NO: 44) | |
| 1 | MALWRPSDNT VYLPPPSVAR VVNTDDYVTR TSIFYHAGSS RLLTVGNPYF RVPAGGGNKQ |
| 61 | DIPKVSAYQY RVFRVQLPDP NKFGLPDNSI YNPETQRLVW ACVGVEIGRG QPLGVGLSGH |
| 121 | PFYNKLDDTE SSHAATSNVS EDVRDNVSVD YKQTQLCILG CAPAIGEHWA KGTACKSRPL |
| 181 | SQGDCPPLEL KNTVLEDGDM VDTGYGAMDF STLQDTKCEV PLDICQSICK YPDYLQMSAD |
| 241 | PYGDSMFFCL RREQLFARHF WNRAGTMGDT VPPSLYIKGT GMRASPGSCV YSPSPSGSIV |
| 301 | TSDSQLFNKP YWLHKAQGHN NGVCWHNQLF VTVVDTTRST NLTICASTQS PVPGQYDATK |
| 361 | FKQYSRHVEE YDLQFIFQLC TITLTADVMS YIHSMNSSIL EDWNFGVPPP PTTSLVDTYR |
| 421 | FVQSVAITCQ KDQLYQTCKA TGTCPPDVIP KVAAPAENKD PYDKLKFWNV DLKEKFSLDL |
| 481 | DQYPLGRKFL VQ |
| HPVchim10 | |
| (SEQ ID NO: 45) | |
| 1 | MSLWLPSEAT VYLPPVPVSK VVSTDEYVAR TNIYYHAGTS RLLAVGHPYF PIKKPNNNKI |
| 61 | LVPKVSGLQY RVFRIHLPDP NKFGFPDTSF YNPDTQRLVW ACVGVEVGRG QPLGVGISGH |
| 121 | PLLNKLDDTE NASAYAAGGL GIGTGSGTGG RTGYVPLNAG VDNRECISMD YKQTQLCLIG |
| 181 | CKPPIGEHWG KGSPCTNVAV NPGDCPPLEL INTVIQDGDM VDTGFGAMDF TTLQANKSEV |
| 241 | PLDICTSICK YPDYIKMVSE PYGDSLFFYL RREQMFVRHL FNRAGAVGEN VPDDLYIKGS |
| 301 | GSTANLASSN YFPTPSGSMV TSDAQIFNKP YWLQRAQGHN NGICWGNQLF VTVVDTTRST |
| 361 | NMSLCAAIST SETTYKNTNF KEYLRHGEEY DLQFIFQLCK ITLTADVMTY IHSMNSTILE |
| 421 | DWNFGLQPPP GGTLEDTYRF VTSQAIACQK HTPPAPKEDP LKKYTFWEVN LKEKFSADLD |
| 481 | QFPLGRKFLL Q |

We claim:

1. A human papilloma virus (HPV) type 18 L1 polypeptide or fragment thereof comprising at least one peptide comprising an epitope of an L2 polypeptide inserted within the HPV L1 polypeptide.

2.-4. (canceled)

5. A polypeptide as claimed in claim 1 wherein the HPV L1 protein comprises a C-terminal deletion of one or more amino acids.

6. A polypeptide as claimed in claim 1, wherein the at least one L2 peptide is inserted within an exposed region of the L1 polypeptide.

7. A polypeptide as claimed in claim 1, wherein the at least one L2 peptide is inserted in the DE Loop of the L1 protein.

8.-12. (canceled)

13. A polypeptide as claimed in claim 1, wherein the at least one L2 peptide is inserted within the C terminus of the L1 polypeptide.

14. (canceled)

15. A polypeptide as claimed in claim 1, comprising two or more L2 peptides inserted within the L1 polypeptide.

16. A polypeptide as claimed in claim 15, wherein the two or more L2 peptides are inserted at different sites.

17. A polypeptide as claimed in claim 16, wherein a first L2 peptide is inserted into the DE loop and a second L2 peptide is inserted into the C terminus of the L1 polypeptide.

18. A polypeptide as claimed in claim 15, wherein the two or more L2 peptides are different.

19. A polypeptide as claimed in claim 1, wherein the at least one L2 peptide comprises at least 8 contiguous amino acids of a native L2 polypeptide.

20. (canceled)

21. (canceled)

22. A polypeptide as claimed in claim 1, wherein the at least one L2 peptide is selected from the group selected of:
a peptide comprising amino acid residues 17-36 of an HPV L2 polypeptide;
a peptide comprising amino acid residues 56-75 of an HPV L2 polypeptide;
a peptide comprising amino acid residues 96-115 of an HPV L2 polypeptide; and
a peptide comprising amino acid residues 108-120 of an HPV L2 polypeptide.

23. A polypeptide as claimed in claim 1, wherein the at least one L2 peptide consists of amino acids 17-36 of HPV type 33 L2 and/or amino acids 56-75 of HPV type 58 L2.

24.-30. (canceled)

31. A polypeptide as claimed in claim 1, wherein the at least one L2 peptide comprises two or more L2 peptides.

32.-34. (canceled)

35. A polypeptide of claim 1, wherein the L2 peptide comprises at least 8 contiguous amino acids, which at least 8 contiguous amino acids comprise a sequence identical to the L2 polypeptides of at least two HPV types.

36.-52. (canceled)

53. A capsomer comprising a polypeptide as claimed in claim 1.

54. A virus like particle (VLP) comprising a polypeptide as claimed in claim 1.

55. An immunogenic composition comprising a polypeptide, capsomer or VLP as claimed in claim 1, and a pharmaceutically acceptable excipient, diluent or carrier.

56. The immunogenic composition as claimed in claim 55, further comprising an adjuvant.

57. The immunogenic composition as claimed in claim 56, wherein the adjuvant comprises an aluminium salt.

58. (canceled)

59. The immunogenic composition as claimed in claim 57, additionally comprising 3D-MPL.

60. A nucleic acid molecule encoding a polypeptide as claimed in claim 1.

61.-64. (canceled)

65. A method for producing a human papilloma virus (HPV) type 18 L1 polypeptide or fragment thereof comprising at least one peptide comprising an epitope of an L2 polypeptide inserted within the HPV L1 polypeptide, comprising introducing an expression vector comprising a nucleic acid of claim 60 into a cell, and replicating the cell under conditions whereby the polypeptide is produced.

66. An immunogenic composition comprising:
(i) at least one virus like particle (VLP) comprising a human papillomavirus (HPV) L1 polypeptide or fragment thereof; and
(ii) at least one chimeric polypeptide comprising a human papillomavirus (HPV) L1 polypeptide or fragment thereof comprising at least one peptide comprising an epitope of an L2 polypeptide inserted within the HPV L1 polypeptide.

67. An immunogenic composition as claimed in claim 66, wherein the VLP of (i) consists of L1 polypeptide or fragment thereof.

68.-77. (canceled)

78. An immunogenic composition as claimed claim 66, wherein each VLP and/or chimeric polypeptide is present in an amount between 10 and 50 μg per human dose.

79. (canceled)

80. An immunogenic composition as claimed in claim 66, further comprising a pharmaceutically acceptable excipient, diluent or carrier.

81. An immunogenic composition as claimed in claim 80, further comprising an adjuvant.

82. The immunogenic composition as claimed in claim 81 wherein the adjuvant comprises an aluminium salt.

83. (canceled)

84. The immunogenic composition as claimed in claim 82, additionally comprising 3D-MPL.

85. A method of preparing an immunogenic composition, the method comprising combining
(i) at least one human papillomavirus (HPV) L1 virus like particle (VLP), with
(ii) at least one chimeric polypeptide comprising a human papillomavirus (HPV) L1 polypeptide or fragment thereof comprising at least one peptide comprising an epitope of an L2 polypeptide inserted within the HPV L1 polypeptide, and
(iii) a pharmaceutically acceptable diluent or carrier and optionally
(iv) an adjuvant, to produce the immunogenic composition according to claim 80.

86. A method for inducing antibodies against HPV in humans comprising administering to a human an immunogenic composition according to claim 66.

87. The method of claim 86, wherein inducing antibodies against HPV prevents, ameliorates or treats HPV infection or disease.

88. (canceled)
